(19) United States
(12) Patent Application Publication
(10) Pub. No.: US 2021/0321932 A1
(43) Pub. Date: Oct. 21, 2021

(54) MEDICAL DEVICE AND METHOD FOR DIAGNOSIS AND TREATMENT OF DISEASE

(71) Applicant: NEUROSPRING, Emeryville, CA (US)

(72) Inventor: Mark K. BORSODY, Emeryville, CA (US)

(21) Appl. No.: 17/272,596

(22) PCT Filed: Aug. 28, 2019
(86) PCT No.: PCT/US2019/048671

§ 371 Date: Mar. 1, 2021

Related U.S. Application Data
(60) Provisional application No. 62/816,239, filed on Mar. 11, 2019.
(60) Provisional application No. 62/723,593, filed on Aug. 28, 2018.

(51) Int. Cl. A61B 5/00; G16H 50/20; G16H 50/30; A61B 5/0205

(52) U.S. Cl. CPC A61B 5/4076; A61B 5/7475; A61B 5/7267; G16H 50/20; G16H 50/30; A61B 5/6891; A61B 5/0022; A61B 5/681; A61B 5/7275; A61B 5/4058; A61B 5/02055; A61B 5/4848; A61B 5/6831

(57) ABSTRACT

A medical device comprising a memory, a processor communicably coupled to the memory, and the processor configured to execute instructions to evaluate one or more patient data inputs relating to a first specific disease, compare the one or more data inputs to a set of values from at least one database using at least one computational algorithm, train the at least one computational algorithm for estimating a diagnosis of the patient based on the first specific disease, determine a first diagnostic score for the patient for the specific disease using the at least one computational algorithm, diagnose the patient as having the specific disease when the first diagnostic score for the first specific disease is above a first value, and provide the diagnosis for the patient as an output.

### CROSS REFERENCE TO RELATED APPLICATIONS/PRIORITY

The present invention claims priority to U.S. Provisional Patent Application No. 62/723,593 filed Aug. 28, 2018, and U.S. Provisional Patent Application No. 62/816,239 filed Mar. 11, 2019, and Patent Cooperation Treaty Application Number PCT/US2019/048,671 filed Aug. 28, 2019, which are all incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

Proper diagnosis of patients is required for medical professionals to prescribe and administer proper treatment of a patient's condition. Some conditions can prevent difficulty in diagnosing accurately, especially at first encounter with the patient. This problem is exacerbated when the condition has a limited window of treatment efficacy, such as with stroke and heart attack, for example. Emergency treatments for different types of acute strokes, specifically ischemic strokes, such as using ‘clot-busting’ recombinant tissue plasminogen activator (rtPA) are rarely given, because of the time it takes to diagnose the conditions Even though stroke is the leading cause of disability and the second most common cause of death globally, and that in the US, nearly 800,000 cases of stroke occur each year, costing approximately $37 billion in healthcare expenses, there 17+ million cases of stroke annually of which 5.7 million are fatal, a rapid and effective diagnostic and treatment tool has not been made available to medical professionals. Neurological emergencies such as acute stroke cause time-dependent brain damage. For the foregoing reasons, there is a pressing, but seemingly irresolvable need for a rapid and effective device and method for the diagnosis and treatment of diseases.

### SUMMARY

Wherefore, it is an object of an embodiment of the present invention to overcome the above-mentioned shortcomings and drawbacks associated with the current technology.

Ischemic stroke currently can be treated in pre-hospital with rtPA. By delivering the patient to the hospital with the diagnosis of acute stroke established, the patient then only needs routine neuroimaging to establish the final diagnosis which leads to IV rtPA treatment. Establishing a diagnosis on site with the disclosed device, eliminates the need for in-hospital physician evaluation, saving on-average 83 minutes of time to IV rtPA treatment, in a situation where earlier treatments are strongly correlated with more successful outcomes. This time saved translates into 37% increase in rtPA effectiveness and 3 times the number of treated patients. Due to its scalability, the medical device disclosed herein could be used in, for example, approximately 81,000 ambulances in the U.S. and 178,000 ambulances in Europe.

According to one embodiment, the disclosed artificial intelligence diagnostic (AID) medical device is a cloud-based artificial neural network with natural language and visual recognition/computer vision that can diagnose neurological emergencies by direct communication with the patient. Once the diagnosis is finished, the medical device will then convey the treatment information to medical personnel and guide the patient's transportation to the correct hospital. Patients who have uncertain diagnoses may be referred to an on-call neurologist for evaluation.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life, and further may include a disordered or incorrectly functioning organ, part, structure, or system of the body resulting from the effect of genetic or developmental errors, infection, poisons, nutritional deficiency or imbalance, toxicity, or unfavorable environmental factors; illness; sickness; ailment.

The presently disclosed invention relates to methods, systems, and medical devices comprising a memory, a processor communicably coupled to the memory, and the processor configured to execute instructions to evaluate one or more patient data inputs relating to a first specific disease, compare the one or more data inputs to a set of values from at least one database using at least one computational algorithm, train the at least one computational algorithm for estimating the diagnosis of the patient based on the first specific disease, determine a first diagnostic score for the patient for the specific disease using the at least one computational algorithm, diagnose the patient as having the specific disease when the first diagnostic score for the first specific disease is above a first value, and display or provide the diagnosis for the patient as an output. According to a further embodiment the processor is further configured to execute instructions to diagnose the patient as not having the specific disease when the diagnostic score is below a second value, with the second value being below the first value. According to a further embodiment the processor is further configured to execute instructions to diagnose the patent as inconclusive when the diagnostic score is between the first value and the second value. According to a further embodiment the medical device further comprises one of a mirror measuring at least one square foot of reflective area, a clock, a refrigerator, a toilet, a chair, a bed, a television, a microwave oven, a floor standing or counter electric light, and a light fixture attached to the ceiling. According to a further embodiment the medical device further comprises straps to allow the medical device to be worn on the wrist. According to a further embodiment the data inputs are one of demographic data, symptoms, medical history elements, examination findings, and/or diagnostic testing results, or some combination thereof. According to a further embodiment the data inputs are entered by one of a patient, a third party, and are automatically acquired by the medical device. According to a further embodiment the medical device initiates interaction with the patient with a spoken prompt. According to a further embodiment the likelihood of diagnosis of a specific disease is increased when a positive symptom of that disease is present, and the likelihood of the diagnosis of the specific disease is decreased when separate symptom of a mimic disease is present. According to a further embodiment the first specific disease is one of a neurological abnormality, congestive heart failure, asthma, myocardial infarction, and an infection. According to a further embodiment the specific disease is a neurological abnormality includes one of acute ischemic stroke, transient ischemic attack, seizure, demyelinating diseases, multiple sclerosis, traumatic brain injury, and brain tumor, or some combination of each. According to a further embodiment medical device automatically evaluates a patient for an initial sign of one or more the specific diseases of conditions, and automatically triggers a more comprehensive evaluation when the initial sign is detected. According to a further embodiment one of abnormal body temperature is assessed as an initial sign of infection, one of alterations in gait, speech, and extremity movements, or some combination thereof are assessed as initial symptoms and signs of a neurological abnormality, one of breathing rates, interruptions in speech, and both are assessed as initial symptoms and signs of worsening congestive heart failure, one of shortness-of-breath, labored breathing, and both are assessed as initial symptoms and signs of an impending or actual asthma attack, and one of gripping the chest, facial expressions indicative of pain, rapid breathing, flushing, and/or sweatiness, or some combination thereof are assessed as initial symptoms and signs of myocardial infarction. According to a further embodiment when the device diagnoses the patient as having a the first specific disease of condition, the device one of determines an appropriate medication to be taken by the patient and informs the patient, and determines an appropriate medication to be taken by the patient, informs the patient, and then dispenses the medication directly also. According to a further embodiment the processor is further configured to execute instructions to evaluate the patient for a likelihood of a second disease that is a mimic to the first disease, and a diagnosis of the first disease is accompanied by an alert when the likelihood of an alternative diagnosis of the mimic condition is above one of 25%, 50%, 75%, and 90%. According to a further embodiment the at least one computational algorithm includes one or more of an artificial neural network, a support-vector machine (SVM), a Nu-SVM, a linear SVM, a Naive Bayes (NB) algorithm, a Gaussian NB, a multinomial NB computation algorithm, a multiclass SVM, a directed acyclic graph SVM (DAGSVM), a structured SVM, a least-squares support-vector machine (LS-SVM), a Bayesian SVM, a transductive support-vector machine, a support-vector clustering (SVC), a classification SVM Type 1 (C-SVM classification), a classification SVM Type 2 (nu-SVM classification), a regression SVM Type 1 (epsilon-SVM regression), and a regression SVM Type 2 (nu-SVM regression). According to a further embodiment the processor is further configured to send the diagnosis to a medical facility via a wired or wireless network, and the medical device further comprises means to communicate the diagnosis via the network. According to a further embodiment the processor is further configured to execute a second computational algorithm to determine a second diagnostic score for the patient when the first diagnostic score is determined to be one of below the first value, below the first value and above the second value, and below both the first value and the second value. According to a further embodiment the processor is further configured to execute a plurality of computational algorithms, each using data from a plurality of databases, determine a diagnostic score for the patient for the first specific disease for each of the computational algorithms, and diagnose the patient as having the specific disease when one of the majority and all of the computational algorithms diagnostic scores for the specific disease is above the first value. According to a further embodiment the processor is further configured to input one or more syndrome elements encompassed by a historical definition of a classic syndrome related to the first specific disease, allocate syndrome elements points proportionate to a known or documented prevalence in a population of patients confirmed to have the classic syndrome, determine if the patient has the syndrome elements, calculate a probability of diagnosis of the patient having the classic syndrome as a ratio of total number of points representing the syndrome elements identified for the patient divided by a total number of points of all the syndrome elements encompassed by the historical definition of the classic syndrome, and input the probability of diagnosis of the classic syndrome as a data input relating to the first disease. According to a further embodiment the first computational algorithm and second computational algorithm are part of a plurality of computational algorithms that refine a diagnosis for the patient in a serial manner. According to a further embodiment the first computational algorithm bases its calculations upon a common set of data inputs from multiple databases, and wherein the second computational algorithm bases its calculations upon all the data inputs from a single database. According to a further embodiment the second computational algorithm is chosen from a plurality of computational algorithms each basing its calculations upon all the data inputs from separate databases, and wherein the second computational algorithm selection is based on similarity between the patient's data inputs and the data inputs of the database used by the second computational algorithm.

According to an even further embodiment, the presently disclosed invention relates to devices, systems and methods comprising generating a value representing a syndrome associated with a medical condition, wherein a syndrome may be a grouping of symptoms, medical history elements, examination findings, and diagnostic testing results that are associated with the medical condition, generating a set of biometric values representing a patient, providing each of the value representing the syndrome and the set of biometric values to a machine learning system to provide an output value indicative of the likelihood that the patient is experiencing the medical condition, and generating an output derived from the output value to a user.

Various objects, features, aspects, and advantages of the present invention will become more apparent from the following detailed description of preferred embodiments of the medical device, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a schematic view of external symptoms and internal neural damage from a classic ischemic stroke syndrome termed the Wallenberg syndrome. Damage in the lateral part of the medulla (the darker portion on the left of the medulla cross section, as shown) compromises certain neuroanatomical structures that then produce a unique combination of symptoms and physical exam abnormalities. The precipitating damage is reliably caused by occlusion of a specific artery, also known as an ischemic stroke;

FIG. 2 is a table summarizing stroke mimics, exclusive of hemorrhagic stroke conditions (intracranial hemorrhage, subarachnoid hemorrhage);

FIG. 3 is a flow chart of domains of computational algorithms employed serially in the diagnostic evaluation of a patient according to an embodiment of the presently claimed invention. In this example of one embodiment of the medical device, a patient with a possible neurological emergency is evaluated by a set of computational algorithms operating in a logically sequential order to refine the diagnosis, for the purpose of identifying ischemic stroke patients suitable for emergency treatment. “Stroke mimics” encompass neurological conditions often mistaken for stroke, such as seizure. AI=artificial intelligence. CA=computational algorithm. TIA=transient ischemic attack;

FIGS. 4A-4D are potential steps involved for point-based weighting/probability calculation for classic syndrome which may be used as a discrete data input for an artificial intelligence-based diagnostic medical device embodiment of the disclosed medical device. FIGS. 4A-4D describe the logic for employing a point-based assessment for identification of classic syndromes such as the Wallenberg ischemic stroke syndrome;

FIG. 5 shows data acquisition tasks (left) of the patient interface of the device will be adjustably sequential based on the importance of that data input. One or more computational algorithm then processes the data inputs (right), the strongest of which may be key definitions. Using the example of a stroke-diagnosing device, key definitions would include acuity of onset, persistence of symptoms, classic stroke syndromes, and stroke mimics (gray box around the top left of center of the figure). Key definitions may require examination findings as well as symptoms and precipitating events to establish; other data inputs may be influential albeit to a lesser degree. With experience, the weights of data inputs (W) are refined to improve diagnostic accuracy versus the ‘gold standard’ physician diagnosis. The calculated probability (netj) then determines the diagnosis, e.g., of acute ischemic stroke (AIS), which can lead to a treatment decision. EMS=emergency medical services; EKG=electrocardiogram; rtPA=tissue plasminogen activator.

FIG. 6 shows a specific example of a tiered multi-level analytic device process having a primary computational algorithm group and three secondary computational algorithm groups, wherein the primary computational algorithm(s) initially evaluates a patient and, if unable to arrive at a diagnosis, engages, employs, or defers to a secondary computational algorithm(s) that undertakes different evaluations of the patient for the purpose of detecting a diagnosis;

FIG. 7 shows a flowchart illustrating an example of a process for domains of computational algorithms employed serially in the diagnostic evaluation of a patient according to an embodiment of the presently claimed invention;

FIG. 8 shows a schematic diagram of a serial method of utilizing multiple databases and computational algorithms to calculate a patient diagnosis;

FIG. 9 shows a schematic diagram of a group method of utilizing multiple databases and computational algorithms to calculate a patient diagnosis;

FIG. 10 shows a flowchart illustrating an example of a process for domains of computational algorithms employed serially in the diagnostic evaluation of a patient according to an embodiment of the presently claimed invention;

FIG. 11 shows an example of a neural network architecture;

FIG. 12 shows a specific schematic example of a neural network having four neural network layers;

FIG. 13 shows a schematic example of a computing device according to embodiments of the disclosed subject matter;

FIG. 14 shows a block diagram illustrating an example of the medical device which includes multiple sensors coupled to a neural network through an interface according to embodiments of the disclosed subject matter;

FIG. 15 shows potential steps involved in a further embodiment for point-based weighting/probability calculation for a classic syndrome as a discrete data input for an artificial intelligence-based diagnostic medical device embodiment of the disclosed medical device;

FIGS. 16 and 17 show a specific example a middle aged African American male who is from New York City, and methods to choose a preferable diagnostic computational algorithm based on the patient's demographic features similarities to patient records of other patients in databases that trained the computational algorithms, using non-geographic demographic similarities (FIG. 16), and geographic similarities (FIG. 17); and

FIGS. 18 and 19 are respectively a schematic representation of elements in an embodiment of the diagnostic medical device (FIG. 18) and a swim diagram showing the flow of processes through the elements in the functioning of the device (FIG. 19). API=application programming interface; LUIS=language understanding intelligence service; CA=computational algorithm; NLP=natural language processing.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

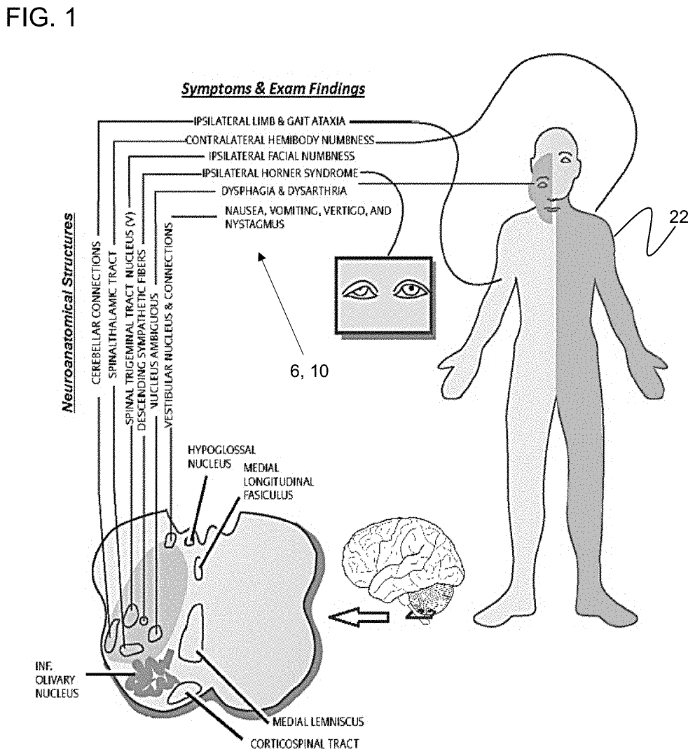

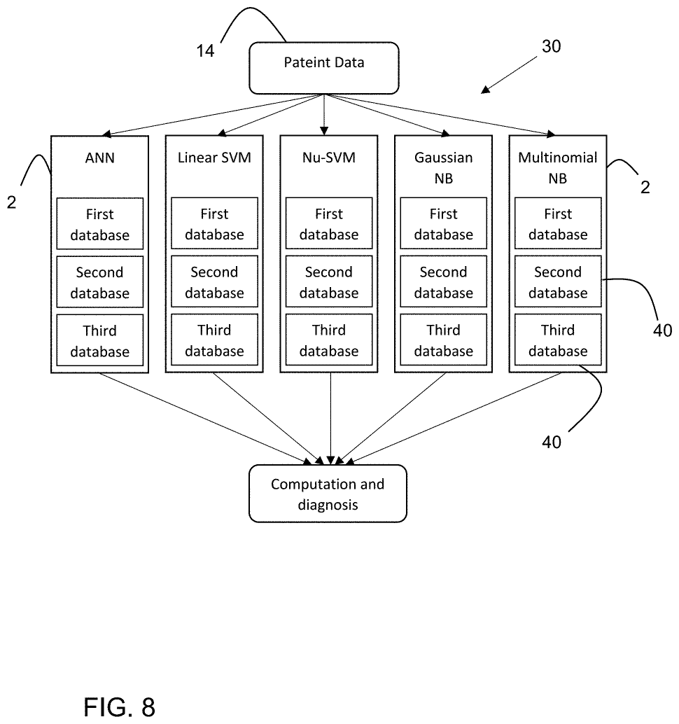

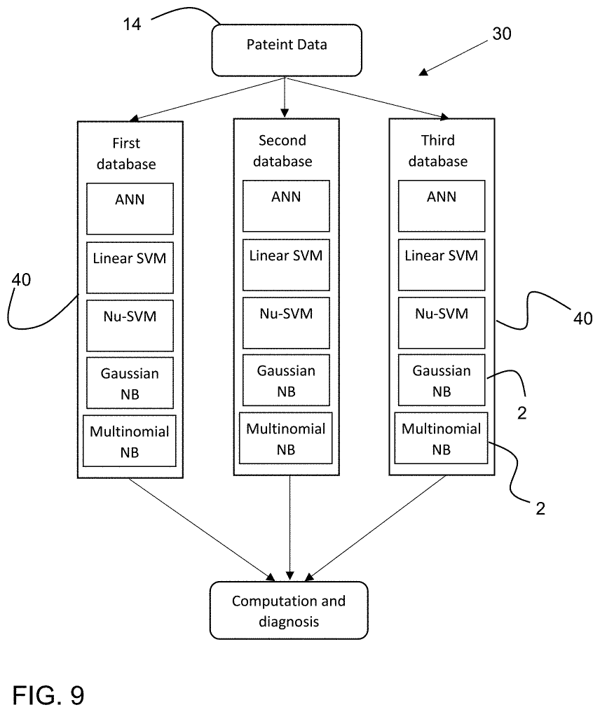

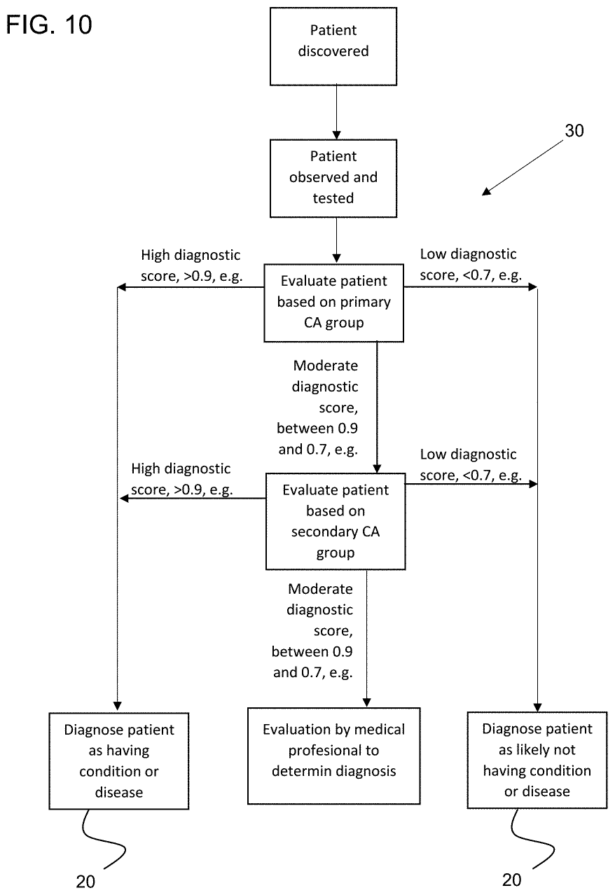

Turning now to FIGS. 1-19, a brief description concerning the various components of the present invention will be discussed. The inventors disclose that computational algorithms 2 (CA), such as artificial neural networks (ANNs), may be used as part of a medical device 3 to predict the diagnosis 20 of disease 4 based on one or more individual symptoms 6, medical history elements 8, examination findings 10, and/or diagnostic testing results 12 (collectively referred to as “data inputs 14”), preferably with each data input 14 receiving its own predictive weight 16 for analysis.

The inventor has observed that certain diseases 4 may lend themselves to identification based upon groupings of highly concurrent symptoms 6, i.e., as a syndrome 18. While a technical definition of “syndrome 18” includes only symptoms 6, the term is used herein to include a broader, more colloquial sense as a grouping of a multiplicity of individual data inputs 14 including, for example, symptoms 6, medical history elements 8, examination findings 10, and/or diagnostic testing results 12. This broader definition is more relevant to medical practice.

The diagnosis 20 of certain diseases 4 in the neurological realm in particular may be predicted by identification of a syndrome 18, which itself can serve as a single data input 14 to computational algorithms 2 or to compliment the diagnostic evaluation of computational algorithms 2. In the clinical neurosciences, focal brain injury typically involves multiple discrete neural structures that are involved in functional networks, wherein the structures and networks are proximate, if not overlapping, in physical space but otherwise might have little or no functional relation. Illustrative examples occur in the brainstem, which serves to connect the large forebrain to the rest of the body directly by the cranial nerves and indirectly through projections into the spinal cord, and within which a majority of non-cognitive neurological functions can be localized. Accordingly, even small focal injuries to the brainstem may be highly symptomatic, but in a manner that is unique based on the portion of the brainstem that is injured. One specific example is a unilateral injury to the lateral portion of the lower part of the brainstem (medulla), causing a grouping of symptoms 6, examination findings 10, and diagnostic testing results 12 known as the Wallenberg syndrome 18, as shown in FIG. 1. The Wallenberg syndrome 18 is strongly associated with occlusion of a vertebral artery or posterior inferior cerebellar artery—thus, it near invariably represents a type of ischemic stroke 4 as being suffered by patients 22 correctly identified as having the syndrome 18—and accordingly would be considered by clinicians as a classic stroke syndrome 18. Other classic neurological syndromes 18 are more strongly associated with other diseases 4 such as seizure, demyelinating diseases such as multiple sclerosis, or traumatic brain injury. A CA 2, such as an ANN, that has as its purpose the diagnosis 20 of ischemic stroke 4 might then identify and thus exclude from the ischemic stroke 4 diagnosis 20 any individuals 22 diagnosed by the medical device 3 with the classic syndromes 18 for these non-ischemic stroke conditions, termed “stroke mimic” conditions 24.

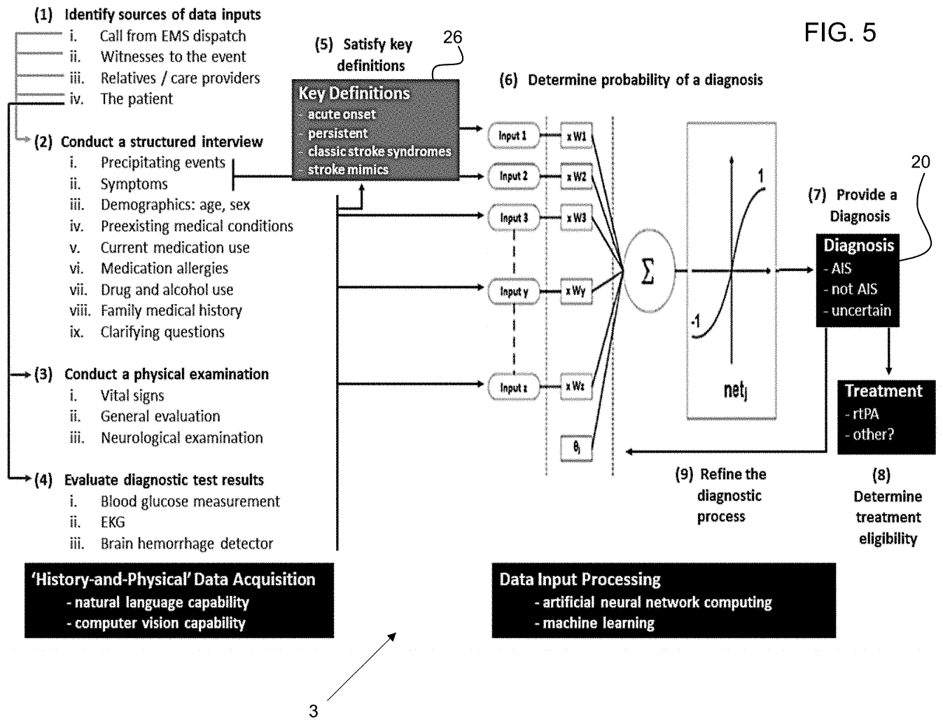

According to one embodiment of the presently claimed medical device 3, is a CA 2, such as an ANN, in which syndromes 18 are evaluated as separate data inputs 14 alongside one or more individual 22 symptoms 6, medical history elements 8, examination findings, and/or diagnostic testing results 12. The weight 16 of the input of a classic syndrome 18 would preferably be high (e.g., 0.9 or greater) and positively predictive for the condition-of-interest. Fulfilling or failing to fulfill the definition of a classic syndrome 18 would be considered, then, a “key definition 26” for the ANN's diagnostic evaluation. Using the example of acute ischemic stroke (AIS), classic stroke syndromes 18 would serve as a key definition 26 for the patient 22 evaluation, as shown in FIG. 5.

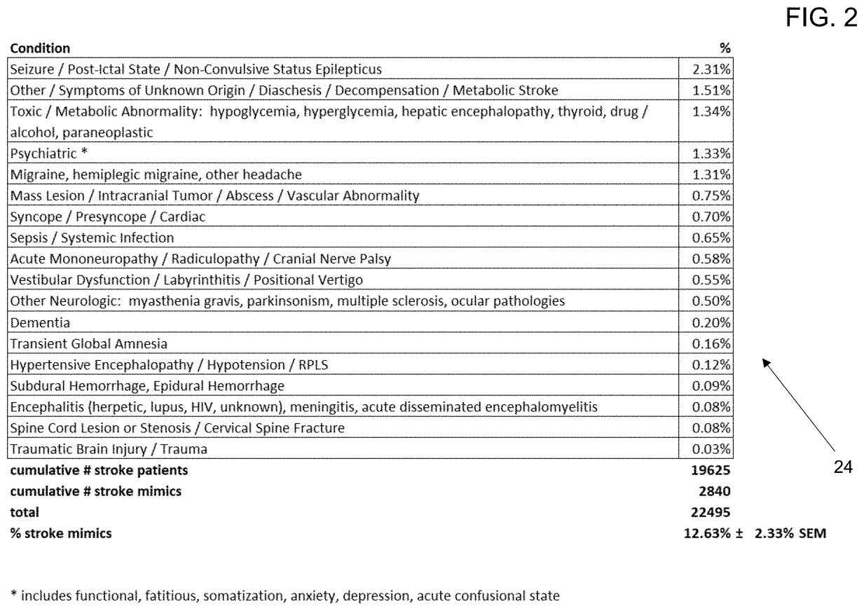

Similarly, other key definitions 26 might include the definitions/diagnostic criteria/characteristics for medical conditions or syndromes that mimic the disease of interest. Using the example of an MS-diagnostic computational algorithm, stroke mimic conditions 24 would include conditions commonly mistaken for MS in the diagnostic setting, such as seizures, hemorrhagic stroke, migraine, or traumatic brain injury. See FIG. 2. Classic stroke mimic syndromes 24 serving as key definitions 26 would preferably have a high magnitude of weight 16 (e.g., 0.9 or greater), but would be negatively predictive of the disease-of-interest 4, here MS.

In further embodiments, the presently claimed invention includes a CA 2, such as an ANN, in which other key definitions 26 would be considered as data inputs 14 for diagnostic purposes. These include, fulfillment or non-fulfillment of pre-determined definitions of (i) acute/sudden onset and (ii) the persistence or resolution of symptoms 6, examination findings, and/or diagnostic testing results.

In a further embodiment, the CA 2 could have magnitude weights 16 of data inputs 14 being relatively ranked as following: key definitions 26 being greater than individual symptoms 6, which are greater than individual examination findings 10, which are greater than medical history elements 8. In some embodiments, diagnostic testing results 12 can serve as key definitions 26, for example, the absence of intracranial hemorrhage on CT scan or other diagnostic test evaluation defining “ischemia” by default or exclusion.

In other embodiments, fulfilment of, or failure to fulfill, a classic syndrome 18 definition is not used by the computational algorithms 2 as a data input 14, but rather confirms the computational algorithm's diagnosis 20 or nullifies the computational algorithm's diagnosis 20, or creates a condition of uncertain diagnosis 20 requiring further evaluation by an on-call physician 28. In other embodiments, satisfaction of the definition of a classic syndrome 18 must first be achieved before data inputs 14 can be evaluated by computational algorithms 2 leading to a diagnosis 20. In still other embodiments, achieving or failing to achieve a classic syndrome 18 definition determines the computational algorithms 2 employed in the patient's evaluation 30, with a plurality of classic syndromes 18 selectively employing computational algorithms 2 from a plurality of computational algorithms 2.

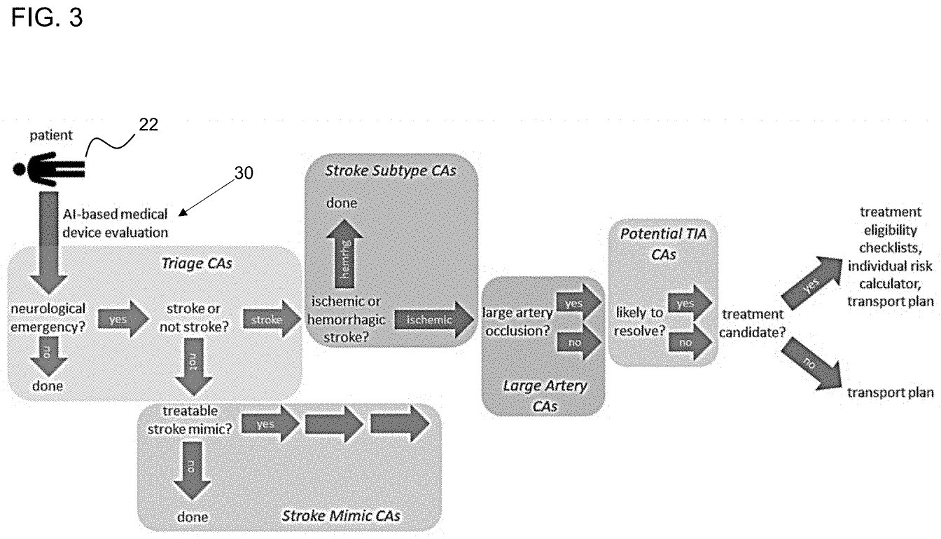

Turning to FIGS. 3-10, a further embodiment of the presently disclosed medical device 3 is shown of artificial intelligence medical devices 3 employing multiple computational algorithms 2.

Artificial intelligence-based diagnostic medical devices 3 (including “software as a medical device 3”) may include computational algorithms 2 structured as, for examples, artificial neural networks, support vector machines, and Bayes algorithms, among others. Individual computational algorithms 2 may be used to predict the diagnosis 20 of diseases 4 based on, for example, data inputs 14 of symptoms 6, medical history elements 8, examination findings 10, and/or diagnostic testing results 12.

According to a further embodiment of the disclosed medical device 3 is an artificial intelligence-based medical device 3, described further below, endowed with one or more computational algorithms 2 that coordinate to predict the diagnosis 20 of a disease 4 by means of, for example, consensus, majority, or other pre-determined threshold. In some embodiments of the medical device 3, if all employed computational algorithms 2 do not agree upon a diagnosis 20, the diagnosis 20 would not be provided to the patient 22 or healthcare provider(s) 28, or would be provided with an alert 32 or warning message, for example that the computational algorithms 2 did not all agree. And if all employed computational algorithms 2 did agree upon a diagnosis 20, in some embodiments, the diagnosis 20 would be provided to the patient 22 or healthcare provider(s) 28. In other embodiments, if at least a majority of the computational algorithms 2 did not agree upon a diagnosis 20, then that diagnosis 20 would not be provided to the patient 22 or healthcare provider(s) 28, or would be provided with an alert 32 or warning message. And if at least a majority of the computational algorithms 2 did agree upon a diagnosis 20, in other embodiments, then that diagnosis 20 would be provided to the patient 22 or healthcare provider(s) 28. In still other embodiments, if one or more computational algorithms 2 acting as “gatekeepers” 34 provide a specific output 38, that would permit other computation algorithm(s) 2 to determine a diagnosis 20, or that would allow other computational algorithm(s) 2 to provide a diagnosis 20, or in the case of a computational algorithm 2 acting as a “poison pill 36” provide a specific output 38 that could prevent other computational algorithms 2 from determining a diagnosis 20 that they might otherwise determine. In such a situation of “gatekeepers” 34 and “poison pills” 36, some computational algorithms 2 or other computer processes may have a specific purpose aside from identifying a primary disease 4 of interest (stroke, for example), e.g., detecting evidence of head trauma, identifying seizure activity on an electroencephalogram, measuring elevated intracranial pressure, or detecting a motor vehicle accident at the emergency scene, mimic conditions 24 which would bring into question a diagnosis 20 of stroke otherwise advanced by different computational algorithms 2 within an artificial intelligence medical device 3. In further embodiments, 2, 3, 4, 5, or more diagnoses 20 could be offered by the computational algorithm 2, with a likelihood of each diagnosis 20 being presented with the respective diagnosis 20, and, preferably, along with the data inputs 14 that suggest each diagnosis 20 and the data inputs 14 that cast doubt on each diagnosis 20, along with, preferably, missing data inputs 14 that could resolve any or all of the diagnoses 20, especially data inputs 14 that are especially indicative of a diagnosis 20 being correct or incorrect, such as gate-keepers 34 and poison pills 36.

Each computational algorithm 2 in the proposed multiplicity of computational algorithms 2 of the artificial intelligence-based diagnostic medical device 3 may employ their own data inputs 14 for training and/or reference. Alternatively, some or all the computational algorithms 2 may base their calculations upon a common set of data inputs 14. In some embodiments of the medical device 3, multiple computational algorithms 2 may have substantially the same original structure and/or code but differ by virtue of having been trained on distinct datasets and therefore having different weights 16 for the same data inputs 14, e.g., as would two initially-identical computational algorithms 2, such as artificial neural networks, that are trained on different patient databases 40. They 2 would have adjusted the respective weights 16 allotted to data inputs 14 based on the predictive weight 16 of the separate data inputs 14 in the separate patient databases 40 for a specific disease 4.

In some embodiments of the proposed machine learning medical device 3, a plurality of computational algorithms 2 and/or analytic processes are engaged in a serial manner to refine a diagnosis 20, as shown in FIG. 5. Refinement of a diagnosis 20 may proceed, e.g., as establishing stroke as the cause of the patient's 22 neurological emergency by one CA 2, then establishing acute/sudden onset of the stroke within a predefined time limit by a second CA 2, then establishing the acute stroke is caused by brain ischemia by a third CA 2, then by determining if the patient 22 is a suitable candidate for treatment with a medication or surgical/endovascular intervention according to indications and contraindications for said treatment(s) by a fourth CA 2. In some embodiments, the coordinated activity of the computational algorithms 2 acts substantially like a decision tree or process flowchart with multiple independent or semi-independent decision-points where decision-making calculations are performed. Other embodiments of the medical device 3 will refer the patient 22 evaluation 30 to a physician 28 or other healthcare provider if its plurality of computational algorithms 2 cannot agree upon a diagnosis 20 for the patient 22 with a high diagnostic value, such as a high probability/degree of certainty. In some embodiments of the medical device 3, the degree of diagnostic accuracy/concordance with a physician's 28 diagnosis 20 achieved by the artificial intelligence-based diagnostic medical device 3 is at least 85%, 90%, 92% 94%, 95%, or 96% for the diagnosis 20 to be considered a high enough probability/degree of certainty.

In still other embodiments of the medical device 3, the involvement of individual 22 computational algorithms 2 is flexible and can be adjusted so that those computational algorithms 2 with high diagnostic confidence will be employed in the patient evaluation 30 and diagnostic decision-making process, whereas other, less effective computational algorithms 2 will only be trained for possible future use upon the patient 22 data and will not contribute to the diagnostic assessment. In such embodiments, the use of specific computational algorithms 2 for diagnostic purposes as part of a plurality of computational algorithms 2 can be changed or adjusted over time depending upon which computational algorithms 2 have the most desirable sensitivity, specificity, positive predictive value, negative predictive value, and/or concordance/agreement rates with other computational algorithms 2. In one such embodiment of the medical device 3, machine learning-capable computational algorithms 2 trained on retrospectively collected patient 22 records are replaced with computational algorithms 2 trained on prospectively-collected patient 22 records. This embodiment may replace the retrospectively-trained computational algorithms 2 with the prospectively trained computational algorithms 2 gradually, for example, in a manner proportionately to the number of patient 22 records upon which they have been trained, or else suddenly upon the prospectively trained computational algorithms 2 achieving a predetermined threshold. In some embodiments, different CAs 2 prospectively collect different data inputs 14 to build prospective databases 40 that they and/or other CAs 2 can employ for training and/or decision-making calculations.

While a technical definition of “syndrome 18” involves only patient 22-reported symptoms 6, herein we use the term in a broader sense as a grouping of a multiplicity of symptoms 6, medical history elements 8, examination findings 10, and/or diagnostic testing results 12 (collectively, “syndrome elements 42”). This broader definition is more appropriate and relevant to medical practice than the technical definition.

Certain diseases are particularly identifiable by the presence of syndromes 18 to the extent of being pathognomonically identifiable by the syndromes 18 (“classic syndromes 18”). The diagnosis 20 of certain medical diseases 4 in the neurological realm are particularly identifiable in this manner. In the clinical neurosciences, focal brain injury/dysfunction typically involves multiple discrete neural structures that critically participate in anatomically distributed functional networks, wherein the neural structures are proximate, if not overlapping, in physical space but otherwise have little or no functional relation. Illustrative examples occur in the brainstem, a portion of the brain that connects the large forebrain to the body directly by the cranial nerves and indirectly through projections into the spinal cord; a majority of non-cognitive neurological functions can be localized in the brainstem. Accordingly, even small focal injuries to the brainstem produce numerous neurological abnormalities in a manner that is unique to the portion of the brainstem that is injured and the nature of the pathophysiological mechanism causing the injury. One example is a unilateral injury to the lateral portion of the lower brainstem (medulla), causing a grouping of syndrome elements 42 known as the Wallenberg syndrome 18, as shown in FIG. 1. The Wallenberg syndrome 18 is strongly associated with occlusion of a vertebral artery or posterior inferior cerebellar artery—thus, it represents a type of ischemic stroke—and accordingly would be considered by clinicians as a “classic stroke syndrome 18” or “classic ischemic stroke syndrome 18” that is sufficiently likely to be ischemic stroke without need for further diagnostic evaluation 30.

Given its high predictive value, then, the presence or absence of a classic syndrome 18 can serve as a single data input 14 to a computation algorithm or plurality of computational algorithms 2 in an artificial intelligence diagnostic medical device 3. However, all syndrome elements 42 may not be present in every patient 22 considered typical for the classic syndrome 18. In order to weight 16 the single data input 14 of a classic syndrome 18 for computational analysis, or to otherwise assess the probability that a patient 22 is suffering from a classic syndrome 18, the inventors disclose a calculation based on the number and prevalence of the syndrome elements 42 present in a given patient 22 versus a population average. For some embodiments of the medical device 3, the inventors disclose a calculation in which: 1) syndrome elements 42, as documented in medical literature, for example, are allocated points proportionate to their known/documented prevalence in a population of patients 22 confirmed to have that classic syndrome 18; 2) a patient 22 under evaluation 30 will be determined to have or not have the syndrome elements 42; 3) the data input's weight 16 or the probability of diagnosis 20 of the classic syndrome 18 is then calculated as the percentage of the points representing the syndrome elements 42 identified in the evaluated patient 22 divided by the total number of points of all that syndrome's 18 elements encompassed by the historical definition of the classic syndrome 18.

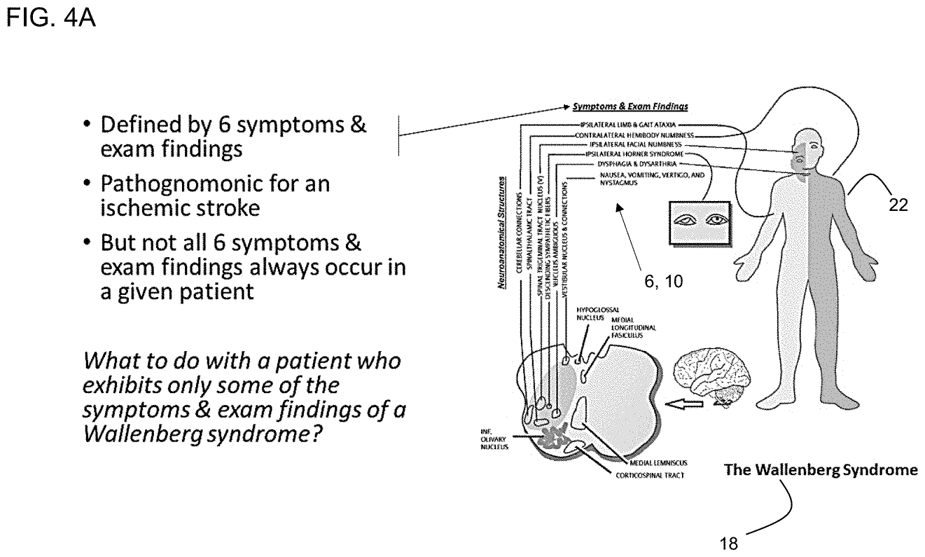

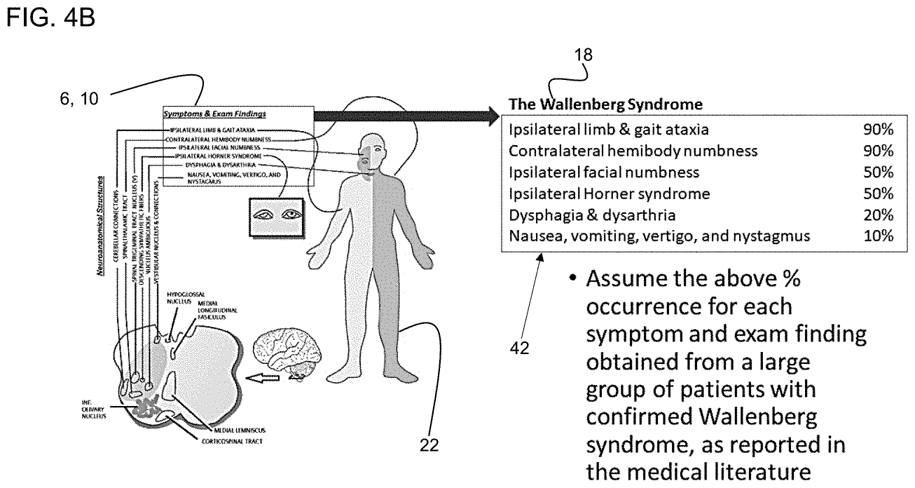

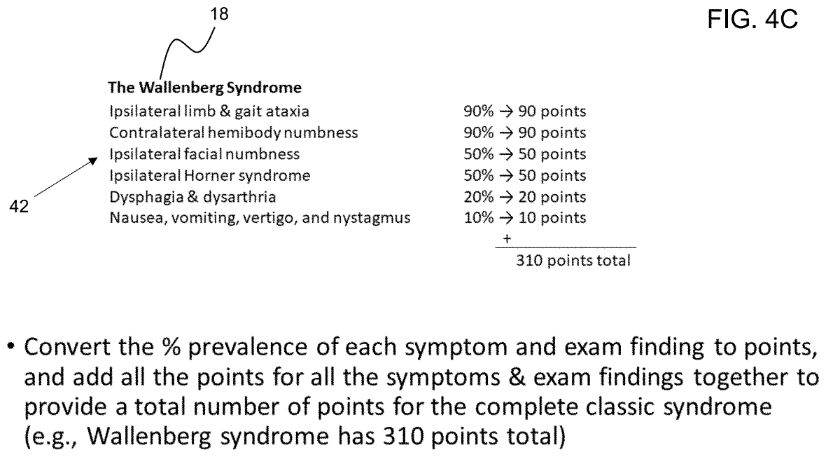

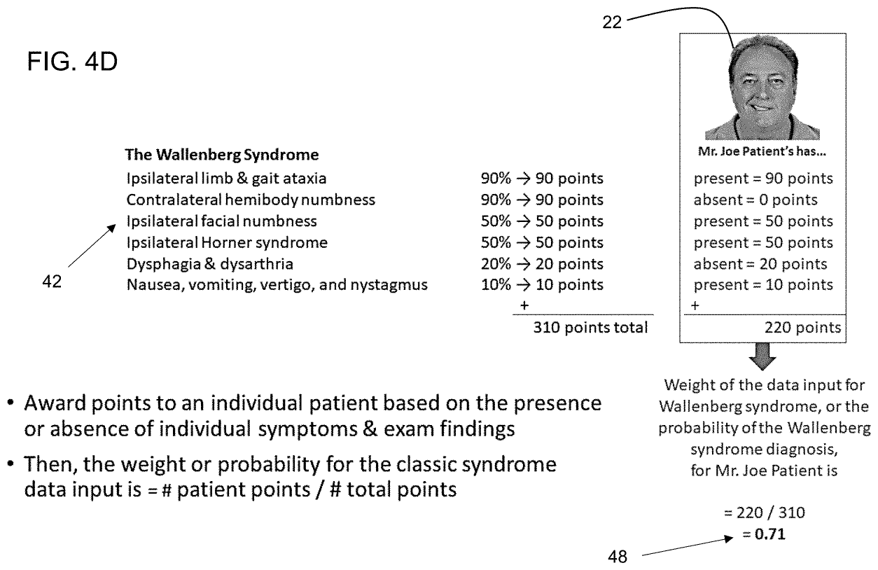

A case example of the point-based assessment for a classic syndrome 18 is shown in FIGS. 4A-4D. Other classic neurological syndromes 18 are more strongly associated with seizure, demyelinating diseases such as multiple sclerosis, brain tumors, or traumatic brain injury, as mimics 24 or counter examples to those for stroke 4. An embodiment of an artificial intelligence-based medical device 3 that has as a purpose the diagnosis 20 of stroke 4 might be required to consider, evaluate, or identify and thus exclude the mimic syndromes 24 (classic syndromes 18 for these non-stroke conditions) or stroke mimics 24. The presence of a mimic syndrome 24 could act as a negative factor, decreasing the likelihood a diagnosis 20 of having a particular disease 4 (such as ischemic stroke). The presence of a mimic syndrome 24 could also act as a full stop, fully preventing a diagnosis 20 of a particular disease 4. Alternatively, or additionally, the medical device 3 could make a diagnosis 20 of a disease 4, but include an alert 32 if the likelihood of an alternative diagnosis 20, such as a mimic condition 24, is above a certain level, such as 15%, 25%, 50%, 75%, or 90%, for example.

In some embodiments of the medical device 3, a patient 22 will be assessed for a classic syndrome 18 only if a pre-determined number or proportion of the syndrome elements 42 are identified during an initial screening of the patient 22, such as, for example, one, two, or three of the most common syndrome elements 42 found in patients 22 diagnosed with the syndrome 18 or one quarter to one half of the syndrome elements 42 found in patients 22 diagnosed with the syndrome 18. The point-based system shown in FIGS. 4A-D can be similarly used to trigger a fuller evaluation of the patient 22 for a classic syndrome 18, wherein the evaluation 30 begins only if a certain proportion or majority of the points representing syndrome elements 42 are identified in a patient 22.

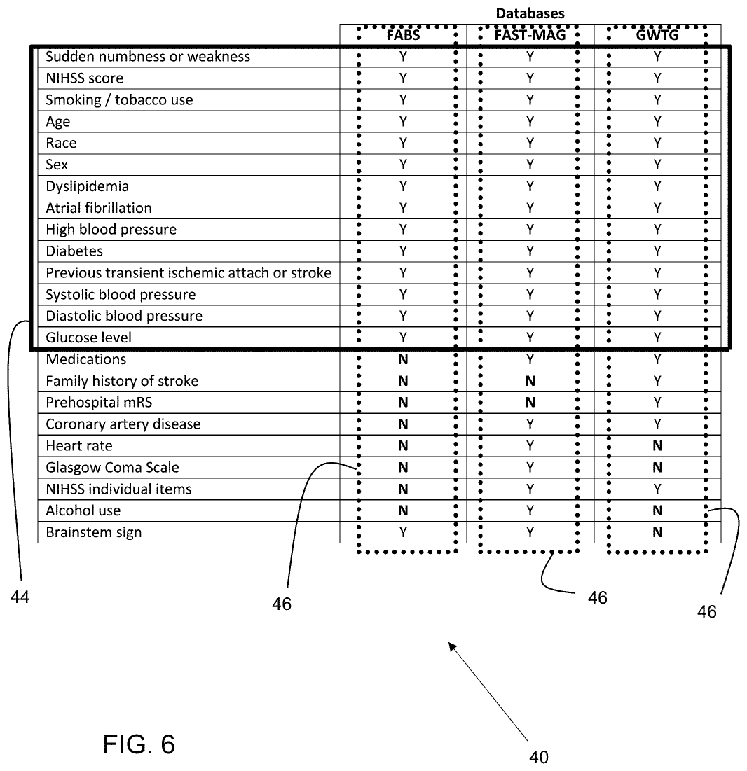

Referring to FIG. 6, a tiered computational algorithm 2 system of the medical device 3 is shown. As shown in the Figure, there may be inconsistent data inputs 14 presented in the various databases 40 composed of records of patients 22 with the same disease 4. In the Figure, “Y” indicates that data exists for the box shown, and “N” indicates that data does not exist for the box shown. Using the example of ischemic stroke 4, many data inputs 14 may be present in all databases 40 (e.g., age, atrial fibrillation) because they are known to strongly predict the given disease 4. Other data inputs 14 may be present in some but not other databases 40, such as alcohol use or family history of stroke. This can present a challenge, namely, how to serially train the disclosed medical device 3 on databases 40 that do not all have the same data inputs 14. Imputation of missing data may be problematic. One method of training the medical device's 3 computational algorithms 2 to increased accuracy in diagnosis 20 is a repetitive cycle of utilizing patient databases 40, including finding a database 40, getting database 40 access, training with the database 40, and then searching for further databases 40. A benefit of this embodiment of a training plan are that it maximizes machine learning capabilities, while a potential disadvantage is that it has a possibility of losing/diluting/overwhelming preexisting training with successive training cycles.

The disclosed medical device 3 may make uses of a variety of databases 40. The FABS database 40 (associated with the FABS scoring system), the FAST-MAG (Field Administration of Stroke Therapy—Magnesium) database 40, and the GWTG (Get With The Guidelines) database 40 are shown just as an example. Alternative and/or additional databases 40 could be used based on availability and applicability for a specific disease.

The inventor discloses multiple embodiments to utilize the various databases 40 to train the medical device 3 and to make diagnosis 20. A first embodiment is a serial train with databases 40 using all data elements and allow for dilution of uncommon data inputs 14. A strength of this embodiment is that it is very simple to implement. A potential weakness is that it may dilute previous training efforts, may neglect the value of hard-to-collect data inputs 14, and may not be feasible given need to constantly impute and delete data inputs 14. A second embodiment is to train only on common data inputs 14 that are available in all databases 40. A strength of this embodiment is that it is very simple to implement. A potential weakness is that it may discard potentially useful data inputs 14, even if they are strongly or only weakly predictive. A third embodiment is to serial train with transfer learning with or without big-to-small database 40 training. A strength of this embodiment is that it protects previous training, and it presumably starts with the most accurate single training estimate first (i.e., the biggest database 40), which limits variance. A potential weakness of this embodiment is that it may be dependent upon having the biggest database 40 available at start and uncertainty around which parts of computational algorithm 2 to freeze during training may mean trial-and-error. A fourth embodiment is to impute values for missing data inputs 14 in all databases 40. A strength of this embodiment is that it is straightforward for some data inputs 14. A potential weakness of this embodiment is that it is potentially confounding, that imputation is not possible with some data inputs 14, that it potentially dilutes the value of previous training, and that there may be numerous missing data inputs 14 leading to potentially large unreliability. A fifth embodiment is to impute missing data inputs 14 for smaller databases 40 based on the largest database 40, and then fuse all of the databases 40 and train on the combined database 40. A strength of this embodiment is that it creates a maximum-sized database 40 and potentially has less unreliability than the fourth embodiment. Potential weaknesses are that it assumes that missing data inputs 14 are of low diagnostic value and that missing data inputs 14 can be imputed. A sixth embodiment sets thresholds for including a data input 14, for example, that the data input 14 must be present in multiple databases 40 and/or must be an accepted disease 4 (e.g., stroke) risk factor. A strength of this embodiment is that it builds on knowledge of established risk factors. A potential weakness is that it potentially eliminates data elements that are neglected or of currently unknown value. A seventh embodiment is to train on the largest database 40, and then validate on smaller databases 40. A strength of this embodiment is that it is not exclusive of other possible designs. A weakness is that it will potentially need to impute missing data inputs 14 in validation databases 40, limiting value of validation process. An eighth embodiment is to combine rare data inputs 14 into groups (e.g., heart rate and body temperature folded into “non-blood pressure vital signs”). A strength of this embodiment is that it is simple. A potential weakness of this embodiment is the potential loss of unique predictive value of the rare data inputs 14.

The CA 2 groups may include one or more ANN, support-vector machines (SVM), including NuSVM and linear SVM, and Naive Bayes (NB) algorithms, including Gaussian NB, and multinomial NB computation algorithms. The CA 2 groups may include multiclass SVM, directed acyclic graph SVM (DAGSVM), structured SVM, least-squares support-vector machine (LS-SVM), Bayesian SVM, transductive support-vector machines, support-vector clustering (SVC), classification SVM Type 1 (also known as C-SVM classification), classification SVM Type 2 (also known as nu-SVM classification), regression SVM Type 1 (also known as epsilon-SVM regression), regression SVM Type 2 (also known as nu-SVM regression). In one embodiment of the device, an NB algorithm and SVM algorithm must agree upon the diagnosis before the diagnosis can be established. In other embodiments of the device, different combinations of 2, 3, 4, 5 or more computational algorithms must agree on the diagnosis before the diagnosis can be established. During a first stage, the device may train a Primary CA group 44 using a pooled database 40 that includes only data inputs 14 common across all databases 40, see FIG. 6, that is, inputs 14 that have representative data in each database 40. During a second stage, or within a second domain, the medical device 3 may train separate groups of CAs 2 using all data inputs 14 from individual databases 40, with each database 40 training its own computational algorithm group (Secondary CA groups 46). The primary and secondary CAs 2 could be the same or different types of algorithms.

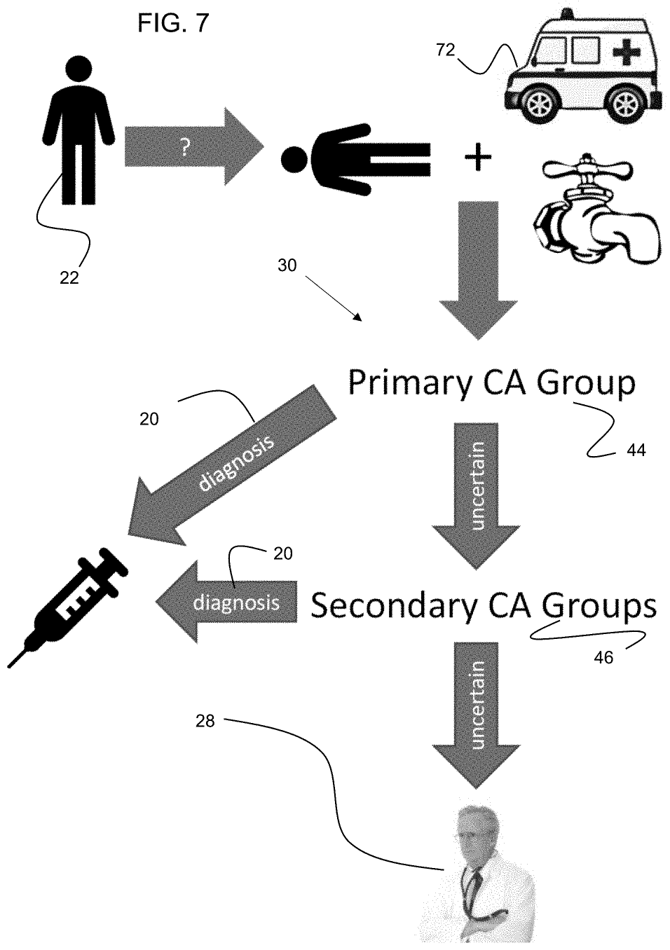

Referring to FIG. 7, in such a scenario, the Primary CA Group 44 gets first attempt to diagnosis 20 based on the common, powerfully-predictive data inputs 14 that are known risk factors for the disease 4. If uncertain, or if the diagnosis score 48 does not reach a first value 50, the patient 22 case may be referred to the Secondary CA groups 46. Additional data inputs 14 may then be obtained by a front-end patient interface 50 of the device, third parties 74, and/or retrieval of patient 22 records, etc., as needed by the Secondary CA group(s) 46. The diagnosis 20 is then reassessed, e.g., using the consensus from the Secondary CA 2 groups as a tiebreaker or by additive probability assessment building upon the Primary CA group 44 calculation. If reevaluation 30 fails to conclusively diagnose 20 a disease 3 is present and/or fails to conclusively diagnose 20 that a disease 3 is not present, the patient 22 may then be referred to a physician 28 (a neurologist in this embodiment) for evaluation 30.

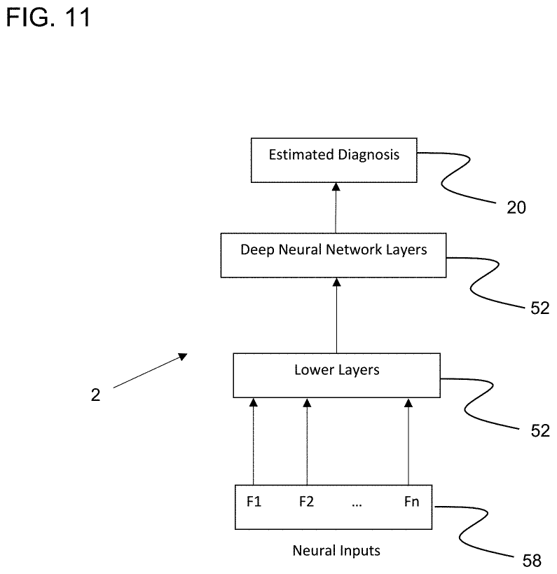

Turning to FIGS. 11-14, embodiments of the disclosed medical device 3 are further discussed. In some embodiments, sensor 62 (microphone and camera, for example) and direct interface 64 acquired device inputs 56 may be passed through a feature extraction module, also called a feature extractor, which transforms the device inputs 56 into “features” 58 that are efficient numerical representations of the device inputs for training the computational algorithm(s) 2. The interface can be a keyboard or a touch screen, for example. In addition to the device input 56 features 58, “labels” 60 for the features may be provided for a symptom. A “label” may include degree of slur in a speech sample, or degree of droop in smile on a first half of a face versus a second half. Other types of indicia of diagnostic symptoms 6 from visual or auditory images/videos or recordings of a patient 22 may also be used, in addition to such as user inputted data 14, including binary or scaled or ranged values entered directly into the medical device 3. This includes “Yes” or “No” binary answers to questions posed by the medical device 3 and range answers from, for example, “0-10” or analog answers, such as real or virtual slider, to questions posed by the medical device 3. The neural network 66 may be trained by receiving, processing, and learning from multiple device inputs 56 and their associated labels 60 or sets of labels 60 to allow for the device to estimate the diagnosis 20 for the patient 22.

In some implementations, a neural network architecture may be constructed with a sufficient number of layers 52 and nodes 60 within each layer 52 such that it can model a diagnosis 20 with enough accuracy when trained with sensor 62 (camera and microphone for example) and user interface (UI) 64 acquired input data 14. FIG. 11 shows an example of a neural network 66 architecture in which features extracted from are provided as neural inputs 68 (f1, f2, . . . fL) to one or more lower layers 52, one or more long-short-term memory (LSTM) layers 52, and one or more deep neural network (DNN) layers 52, to estimate a diagnostic score 48. Various types of neural network layers 52 may be implemented within the scope of the disclosure. For example, alternative or in addition to the DNN layers 52, one or more convolutional neural network (CNN) layers 52 or LSTM layers 52 may be implemented. In some instances, various types of filters such as infinite impulse response (IIR) filters, linear predictive filters, Kalman filters, or the like may be implemented in addition to or as part of one or more of the neural network layers.

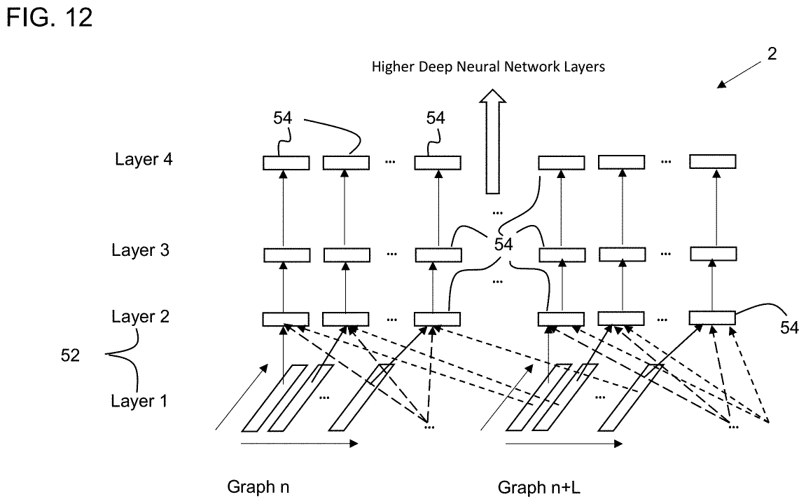

FIG. 12 shows a specific example of one embodiment of a neural network 66 having four neural network layers 52, namely, Layer 1, Layer 2, Layer 3, and Layer 4, for processing features 58 extracted from the device inputs 56. In FIG. 12, two graphs, namely, graph n and graph n+L, are illustrated. It is understood that over a given length of time, device inputs 56 may be represented as multiple graphs, and a magnitude of a graph may represent a diagnostic score 48. For graph n, the first layer 52, that is, Layer 1, includes device inputs for multiple data inputs 14 for each, as illustrated in a first diagnostic score 48. Likewise, for graph n+L, Layer 1 includes device inputs 56 from multiple data inputs 14 for each, as illustrated in a second diagnostic score 48. Additional information on the patient 22 may be included in Layer 1. For changing data inputs 14, Layer 1 of graph n and graph n+L, may include device inputs 56 representing symptoms 6 that vary over time. In an embodiment, with sufficient numbers of nodes 54 or units in Layers 2 and 3, the neural network 66 would be able to gain knowledge or diagnostic accuracy and predict a diagnosis 20.

At least one layer 52 of the neural network 66 may be required to process complex numbers. In one example, the complex numbers may be processed in Layer 2 of the neural network 66. A complex number may be in the form of a real component and an imaginary component, or alternatively, in the form of a magnitude and a phase. In Layer 2 of the neural network, for example, each unit or node 54 may receive complex inputs and produce a complex output. In this example, a neural unit with complex inputs and a complex output may be a relatively straightforward setup for Layer 2. In one example, the net result U within a complex unit is given by: U=Σᵢ Wᵢ Xᵢ+V, where Wᵢ is the complex-valued weight 16 connecting complex-valued inputs, and V is the complex-valued threshold value. In order to obtain the complex-valued output signal, the net result U is converted into real and imaginary components, and these real and imaginary components are passed through an activation function ƒ_{R}(x) to obtain an output ƒₒᵤₜ, given by ƒₒᵤₜ=ƒ_{R} [symbol] (U))+iƒ_{R}( [symbol] (U)); where f r [symbol] (x)=1 1+e−x; x ϵR, for example. Various other complex-value computations may also be implemented within the scope of the disclosure.

In another embodiment, Layer 1 and Layer 2 of the neural network 66 may involve complex computations whereas the upper layers 52, for example, Layer 3 and Layer 4, may involve computations of real numbers. For example, each unit or node 54 in Layer 2 of the neural network 66 may receive complex inputs and produce a real output. Various schemes may be implemented to generate a real output based on complex inputs. For example, one approach is to implement a complex-input-complex-output unit and to make the complex output real by simply taking the magnitude of the complex output: ƒₒᵤₜ=|ƒ_{R} [symbol] (U))+iƒ_{R}( [symbol] (U))|. Alternatively, another approach is to apply the activation function on the absolute value of the complex sum, that is: ƒₒᵤₜ=ƒR(|U|). In another alternative approach, each complex input feature is broken down into either the magnitude and phase components or the real and imaginary components. These components may be regarded as real input features. In other words, each complex number may be regarded as two separate real numbers representing the real and imaginary components of the complex number, or alternatively, two separate real numbers representing the magnitude and phase of the complex number.

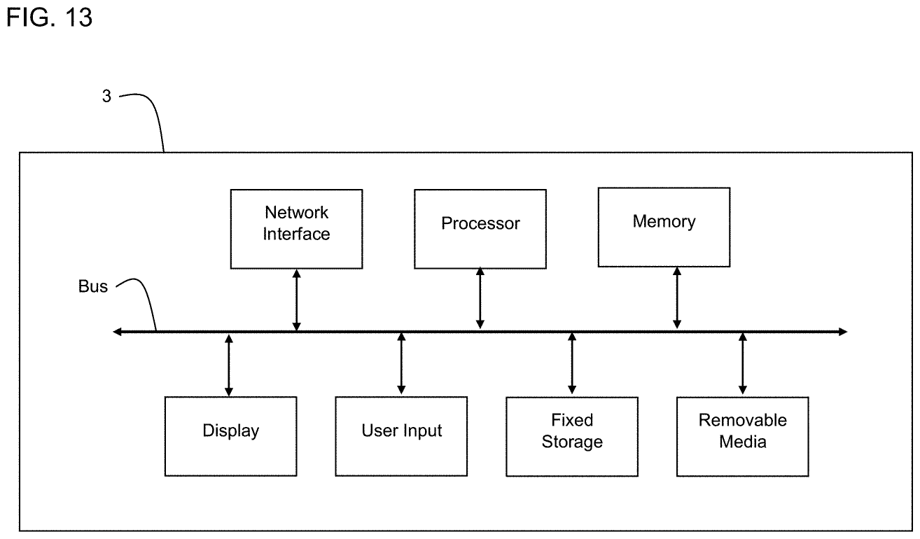

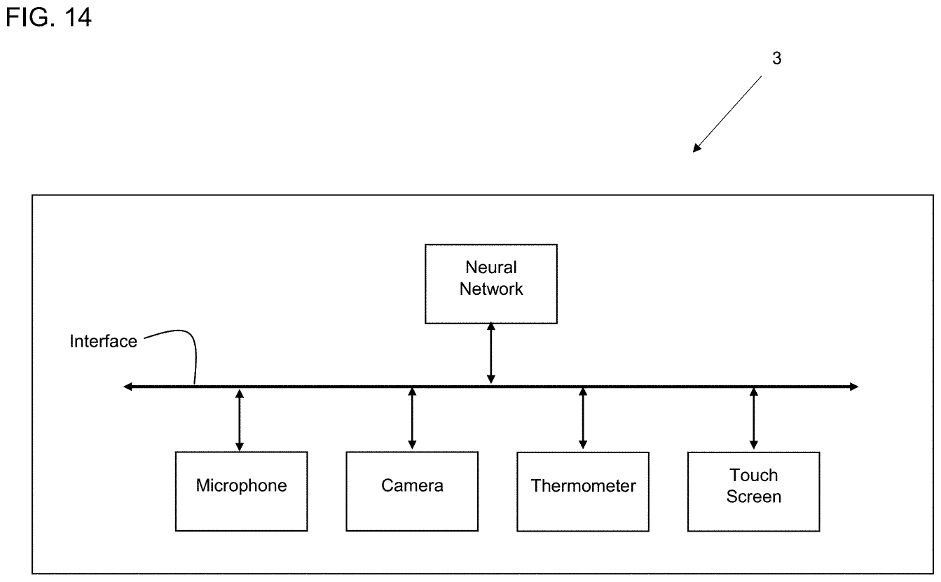

Embodiments of the presently disclosed subject matter may be implemented in and used with a variety of component and network architectures. For example, the medical device 3 neural network 66 as shown in FIG. 14 may include one or more computing devices 70 for implementing embodiments of the subject matter described above. FIG. 13 shows an example of a computing device 70 suitable for implementing embodiments of the presently disclosed subject matter. The computing device 70 may be, for example, a desktop or laptop computer, or a mobile computing device such as a smart phone, tablet, video conferencing/telemedicine system or the like. The computing device 70 may include a bus which interconnects major components of the computer, such as a central processor, a memory such as Random Access Memory (RAM), Read Only Memory (ROM), flash RAM, or the like, a user display such as a display screen, a user input interface, which may include one or more controllers and associated user input devices such as a keyboard, mouse, touch screen (which may be considered part of the interface 64), and the like, a fixed storage such as a hard drive, flash storage, and the like, a removable media component operative to control and receive an optical disk, flash drive, and the like, and a network interface operable to communicate with one or more remote devices via a suitable network connection.

The bus allows data communication between the central processor and one or more memory components, which may include RAM, ROM, and other memory, as previously noted.

Typically, RAM is the main memory into which an operating system and application programs are loaded. A ROM or flash memory component can contain, among other code, the Basic Input-Output System (BIOS) which controls basic hardware operation such as the interaction with peripheral components. Applications resident with the computer are generally stored on and accessed via a computer readable medium, such as a hard disk drive (e.g., fixed storage), an optical drive, floppy disk, or other storage medium.

The fixed storage may be integral with the computer or may be separate and accessed through other interfaces. The network interface may provide a direct connection to a remote server via a wired or wireless connection. The network interface may provide such connection using any suitable technique and protocol as will be readily understood by one of skill in the art, including digital cellular telephone, Wi-Fi, Bluetooth®, near-field, and the like. For example, the network interface may allow the computer to communicate with other computers via one or more local, wide-area, or other communication networks, as described in further detail below.

Many other devices or components (not shown) may be connected in a similar manner (e.g., document scanners, digital cameras and so on). Conversely, all the components shown in FIG. 13 need not be present to practice the present disclosure. The components can be interconnected in different ways from that shown. The operation of a computer such as that shown in FIG. 13 readily known in the art and is not discussed in detail in this application. Code to implement the present disclosure can be stored in computer-readable storage media such as one or more of the memory, fixed storage, removable media, or on a remote storage location.

More generally, various embodiments of the presently disclosed subject matter may include or be embodied in the form of computer-implemented processes and apparatuses for practicing those processes. Embodiments also may be embodied in the form of a computer program product having computer program code containing instructions embodied in non-transitory or tangible media, such as floppy diskettes, CD-ROMs, hard drives, USB (universal serial bus) drives, or any other machine readable storage medium, such that when the computer program code is loaded into and executed by a computer, the computer becomes an apparatus for practicing embodiments of the disclosed subject matter. Embodiments also may be embodied in the form of computer program code, for example, whether stored in a storage medium, loaded into or executed by a computer, or transmitted over some transmission medium, such as over electrical wiring or cabling, through fiber optics, or via electromagnetic radiation, such that when the computer program code is loaded into and executed by a computer, the computer becomes an apparatus for practicing embodiments of the disclosed subject matter. When implemented on a general-purpose microprocessor, the computer program code segments configure the microprocessor to create specific logic circuits.

In some configurations, a set of computer-readable instructions stored on a computer-readable storage medium may be implemented by a general-purpose processor, which may transform the general-purpose processor or a device containing the general-purpose processor into a special-purpose device configured to implement or carry out the instructions. Embodiments may be implemented using hardware that may include a processor, such as a general-purpose microprocessor or an Application Specific Integrated Circuit (ASIC) that embodies all or part of the techniques according to embodiments of the disclosed subject matter in hardware or firmware. The processor may be coupled to memory, such as RAM, ROM, flash memory, a hard disk or any other device capable of storing electronic information. The memory may store instructions adapted to be executed by the processor to perform the techniques according to embodiments of the disclosed subject matter.

In some embodiments, the microphones and cameras as shown in FIG. 14 may be implemented as part of a network of sensors 62. These sensors 62 may include microphones for sound detection and cameras for visual detection, for example, and may also include other types of sensors 62. In general, a “sensor 62” may refer to any device that can obtain information about its environment. Sensors 62 may be described by the type of information they collect. For example, sensor 62 types as disclosed herein may include waveform, chemical emission, motion, smoke, carbon monoxide, proximity, temperature, time, physical orientation, acceleration, location, entry, presence, pressure, light, sound, and the like. A sensor 62 also may be described in terms of the particular physical device that obtains the environmental information. For example, an accelerometer may obtain acceleration information, and thus may be used as a general motion sensor 62 or an acceleration sensor 62. A sensor 62 also may be described in terms of the specific hardware components used to implement the sensor 62. For example, a temperature sensor 62 may include a thermistor, thermocouple, resistance temperature detector, integrated circuit temperature detector, or combinations thereof. A sensor 62 also may be described in terms of a function or functions the sensor 62 performs within an integrated sensor 62 network, such as a smart home environment. For example, a sensor 62 may operate as a security sensor 62 when it is used to determine security events such as unauthorized entry. A sensor 62 may operate with different functions at different times, such as where a motion sensor 62 is used to control lighting in a smart home environment when an authorized user is present, and is used to alert to unauthorized or unexpected movement when no authorized user is present, or when an alarm system is in an “armed” state, or the like. In some cases, a sensor 62 may operate as multiple sensor 62 types sequentially or concurrently, such as where a temperature sensor 62 is used to detect a change in temperature, as well as the presence of a person or animal. A sensor 62 also may operate in different modes at the same or different times. For example, a sensor 62 may be configured to operate in one mode during the day and another mode at night. As another example, a sensor 62 may operate in different modes based upon a state of a home security system or a smart home environment, or as otherwise directed by such a system.

In general, a “sensor 62” as disclosed herein may include multiple sensors 62 or sub-sensors 62, such as where a position sensor 62 includes both a global positioning sensor 62 (GPS) as well as a wireless network sensor 62, which provides data that can be correlated with known wireless networks to obtain location information. Multiple sensors 62 may be arranged in a single physical housing, such as where a single device includes movement, temperature, magnetic, or other sensors 62. Such a housing also may be referred to as a sensor 62 or a sensor 62 device. For clarity, sensors 62 are described with respect to the particular functions they perform, or the particular physical hardware used, when such specification is necessary for understanding of the embodiments disclosed herein.

A sensor 62 may include hardware in addition to the specific physical sensor 62 that obtains information about the environment. The sensor 62 may include an environmental sensor 62, such as a temperature sensor 62, smoke sensor 62, carbon monoxide sensor 62, motion sensor 62, accelerometer, proximity sensor 62, passive infrared (PIR) sensor 62, magnetic field sensor 62, radio frequency (RF) sensor 62, light sensor 62, humidity sensor 62, pressure sensor 62, microphone, weight scale, or any other suitable environmental sensor 62, that obtains a corresponding type of information about the environment in which the sensor 62 is located. A processor may receive and analyze data obtained by the sensor 62, control operation of other components of the sensor 62, and process communication between the sensor 62 and other devices. The processor may execute instructions stored on a computer-readable memory. The memory or another memory in the sensor 62 may also store environmental data obtained by the sensor 62. A communication interface, such as a Wi-Fi or other wireless interface, Ethernet or other local network interface, or the like may allow for communication by the sensor 62 with other devices. The user interface may provide information to or receive input from a user or the sensor 62. The UI 64 may include, for example, a speaker to output 38 an audible alarm when an event is detected by the sensor 62. Alternatively, or in addition, the UI 64 may include a light to be activated when an event is detected by the sensor 62. The user interface may be relatively minimal, such as a limited-output 38 display, or it may be a full-featured interface such as a touchscreen. Components within the sensor 62 may transmit and receive information to and from one another via an internal bus or other mechanism as will be readily understood by one of skill in the art. Furthermore, the sensor 62 may include one or more microphones to detect sounds in the environment. One or more components may be implemented in a single physical arrangement, such as where multiple components are implemented on a single integrated circuit. Sensors 62 as disclosed herein may include other components or may not include all of the illustrative components shown.

Sensors 62 as disclosed herein may operate within a communication network, such as a conventional wireless network, or a sensor 62-specific network through which sensors 62 may communicate with one another or with dedicated other devices. In some configurations one or more sensors 62 may provide information to one or more other sensors 62, to a central controller, or to any other device capable of communicating on a network with the one or more sensors 62. A central controller may be general- or special-purpose. For example, one type of central controller is a home automation network that collects and analyzes data from one or more sensors 62 within the home. Another example of a central controller is a special-purpose controller that is dedicated to a subset of functions, such as a security controller that collects and analyzes sensor 62 data primarily or exclusively as it relates to various security considerations for a location. A central controller may be located locally with respect to the sensors 62 with which it communicates and from which it obtains sensor 62 data, such as in the case where it is positioned within a home that includes a home automation or sensor 62 network. Alternatively, or in addition, a central controller as disclosed herein may be remote from the sensors 62, such as where the central controller is implemented as a cloud-based system that communicates with multiple sensors 62, which may be located at multiple locations and may be local or remote with respect to one another.

Moreover, the smart-home environment may make inferences about which individuals 22 live in the home and are therefore users and which electronic devices are associated with those individuals 22. As such, the smart-home environment may “learn” who is a user (e.g., an authorized user) and permit the electronic devices associated with those individuals 22 to control the network-connected smart devices of the smart-home environment, in some embodiments including sensors 62 used by or within the smart-home environment. Various types of notices and other information may be provided to users via messages sent to one or more user electronic devices. For example, the messages can be sent via email, short message service (SMS), multimedia messaging service (MMS), unstructured supplementary service data (USSD), as well as any other type of messaging services or communication protocols.

A smart-home environment may include communication with devices outside of the smart-home environment but within a proximate geographical range of the home. For example, the smart-home environment may communicate information through the communication network or directly to a central server or cloud-computing system regarding detected movement or presence of people, animals, and any other objects and receive back commands for controlling the ambient lighting accordingly.

In some embodiments of the medical device 3, the medical device 3 regularly evaluates the patient 22 for signs of illness or abnormality which, if detected, may automatically trigger a more comprehensive device evaluation 30 of the patient 22 leading to a diagnosis 20. In one embodiment of the medical device 3, the regular evaluation 30 involves assessment of gait, speech, and extremity movements for signs of a neurological abnormality such as, respectively, a limp, slurred speech, weakness in one arm, or drooping in part of the face. In that embodiment of the medical device 3, detection of such a neurological abnormality will trigger a full assessment of symptoms 6 and examination abnormalities that would be consistent with a focal brain injury such as by stroke. In another embodiment of the device, a patient 22 with known congestive heart failure is assessed for breathing rates or interruptions in speech suggestive of worsening congestive heart failure, at which point the patient 22 will be specifically assessed for his or her condition, potentially including weighing the patient 22. In that embodiment, the device may direct changes in the patient 22's medication such as diuretic dosing use based on the diagnosis 20 of worsening congestive heart failure. In another embodiment of the device, shortness-of-breath or labored breathing would be identified and interpreted by the device as evidence of an impending or actual asthma attack, at which point in time the patient 22 would be informed to use any of a variety of available breathing treatments including inhalers and/or, if the patient 22's condition is sufficiently severe, the device would notify nursing and/or emergency medical services 28 to assist the patient 22. In another embodiment of the device, the device will identify behaviors of the monitored person 22 suggestive of myocardial infarction such as gripping the chest, facial expressions indicative of pain, rapid breathing, flushing, and/or sweatiness, at which point in time the device will confirm other symptoms 6 and examination findings 10 consistent with myocardial infarction, leading to direction of the patient 22 to take emergency treatments for myocardial infarction and notifying an ambulance 72 to retrieve the patient 22 based on the likely diagnosis 20 of myocardial infarction. In another embodiment of the device, a regular thermal scan or point temperature measure of the patient 22 can be used to identify an abnormal body temperature, which will trigger evaluation 30 of the patient 22 by the medical device 3 for symptoms 6 consistent with infection; based on the medical device's 3 diagnosis 20, presumptive antibiotic treatment can be self-administered by the patient 22, or provided to the patient 22 by some third party 74, prior to evaluation 30 of the patient 22 at a physician 28's office or hospital. In additional embodiments, when the medical device 3 determines that a medication is needed by the patient 22, the medical device 3 may dispense the medication directly also. In an additional embodiment, a same medical device 3 may evaluate patients 22 for any or all the above diseases 4.

In some embodiments of the medical device 3, any electronic device equipped with a combination sufficient input devices 76 (such as a microphone, a thermometer, and one or more cameras), output devices 38 (such as speakers, and lights), a processor and/or a memory, for example, can be used exclusively or in coordination to monitor the patient 22. In some embodiments of the medical device 3, the variety of electronic devices are spread throughout the patient's 22 home to monitor the patient 22.

In some embodiments of the medical device 3, the medical device 3 passively assesses the patient 22 for evidence of certain diseases 4. When used in the ambulance 72, the medical device 3 can listen to the report provided by the ambulance/emergency medicine services (EMS) dispatcher and interpret it for indications that the next patient 22 to be seen by the ambulance's crew has a certain disease 4 of interest, such as a stroke. In that situation, for example, the medical device 3 can have the capability of activating itself and conducting a comprehensive evaluation 30 on the patient 22. In other situations, the medical device 3 may request the opportunity to evaluate the patient 22 based on the conversation (either face to face or over the phone, for example) between the patient 22 and an emergency medicine technician or paramedic, and/or upon the physical evaluation 30 of the patient 22 by such an ambulance 72 based healthcare provider.

The first role of one embodiment of the present medical device 3 is to diagnose ischemic stroke in the pre-hospital setting. Based on that diagnosis 20, treatment options may become immediately available. A preferred embodiment of the medical device 3 identifies the resolution of ischemic stroke symptoms 6 and examination findings 10, indicating that the ischemic stroke has resolved and that the patient 22 has had a transient ischemic attack, in which case the medical device 3 will direct the administration of aspirin or other anti-platelet medication to the patient 22 prior to further diagnostic evaluation 30 or arrival at the hospital. The administration of the medications under the medical device 3's direction may be achieved prior to the arrival of any healthcare provider or specialized capabilities, including nurses, paramedics, or ambulances. In a further embodiment, a medical treatment for ischemic stroke, such as a facial nerve stimulator, which is sufficiently safe in the condition of hemorrhagic stroke and thus can be administered to undifferentiated stroke patients 22 without prior neuroimaging evaluations 30, the present medical device 3 can diagnose the patient 22 with stroke and then direct treatment with a transcranial magnetic stimulation (TMS) facial nerve stimulator. In a further embodiment, the medical device 3 can itself provide TMS to the patient 22 after diagnosing the patient 22 with a stroke. In other embodiments, after an initial therapeutic TMS stimulation has been given to the patient, the medical device 3 will assess for improvement in the patient's symptoms 6 and examination abnormalities, and also identify recurrence or new occurrence of symptoms 6 and examination abnormalities that warrant/benefit from repeat therapeutic TMS stimulation.

By establishing the diagnosis 20 of stroke in the ambulance 72 or elsewhere prior to arrival at the hospital, the patient 22 upon arrival at the hospital can be immediately taken to neuroimaging to identify intracranial hemorrhage, which would establish the diagnosis 20 of hemorrhagic stroke and thus preclude treatment with established therapies for ischemic stroke such as intravenous tissue plasminogen activator (rtPA) or endovascular catheter-based procedures. Such a system would bypass the emergency department evaluation 30 and facilitate treatment of ischemic stroke patients 22.

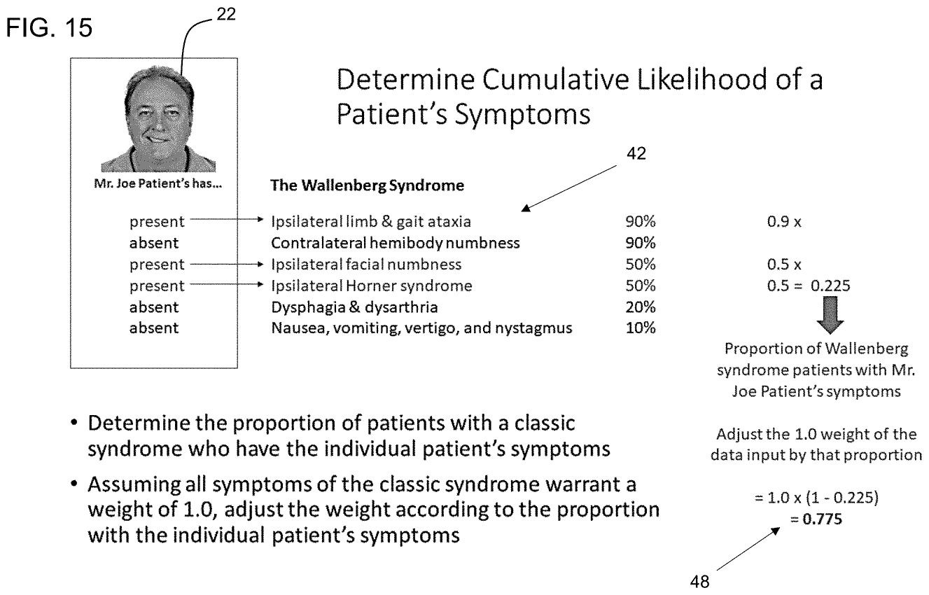

Referring to FIG. 15, a further embodiment for determining the diagnosis of a classic syndrome 18 based on a cumulative likelihood of a patient 22's symptoms 6 and signs are shown. In this embodiment, the proportion of patients 22 with a classic syndrome 18 who have the individual symptoms 6, exam abnormalities 10, or diagnostic test findings 12 (“syndrome 18 components”) are compiled into a database 40 or library. A bias factor may also be given to each syndrome element 42 either equally or unequally, where unequal bias factors may be determined by patient 22-reported survey, impact upon quality-of-life, or the decision of one or more healthcare providers. Assuming, e.g. all syndrome elements 42 of the classic syndrome 18 warrant a bias factor of 1.0, the weight 16 of the data input for the classic syndrome 18 can then be adjusted according to the proportion of the individual patient 22's syndrome elements 42. In this example shown in FIG. 15, ipsilateral limb and gait ataxia, ipsilateral facial numbness, and ipsilateral Horner syndrome 18 are present in the assessed patient 22, and contralateral hemibody numbness, dysphagia and dysarthia, and nausea, vomiting, vertigo, and nystagmus are absent. The present syndrome 18 components have relative proportions of 90%, 50%, 50%, 90%, 20%, and 10%. The device would calculate function of the symptoms 6 and examination findings 10 and the percentage of individuals 22 with the disease who had all the patient 22's syndrome 18 components, by subtracting the product of the probabilities from 1. For example, if a patient 22 has only ipsilateral limb and gait ataxia and contralateral hemibody numbness, and the bias factor for each syndrome element 42=1, then a diagnostic score 48 is 1−(0.9*−0.9)=1−(0.81)=0.19. If all symptoms 6 and examination findings 10 in FIG. 15 are present, a diagnostic score 48 according to this embodiment would will be 0.996. Diagnosis 20 of a specific disease 4 could be made upon achieving a high diagnostic value 78 of the diagnostic score 48, which could be values of greater than 0.80, greater than 0.85, greater than 0.90, greater than 0.95, for example. Diagnostic scores 48 of, for example, between 80% and less than 100% of these high diagnostic values 78 (for example, between 0.64 and 0.80 for embodiments of a high diagnostic value of 0.80, and between 0.72 and 0.90 for embodiments of a high diagnostic value of 0.90) could be flagged as lacking specificity to make or discount a diagnosis 20 of the specific disease, and/or trigger referral to a medical professional for a diagnosis 20. Such moderate diagnostic scores 48 would be termed as having moderate diagnostic values 80 and as being uncertain for the specific disease 4. Other lower end values of the moderate diagnostic values could be 75%, 85%, 90% and 95% of the value for the high diagnostic value 78. Low diagnostic scores 48, for example values below four fifths or 80% of the high diagnosis values 78 (for example, below 0.64 for embodiments of a high diagnostic value of 0.80, and below 0.72 and 0.90 for embodiments of a high diagnostic value of 0.90) could be diagnosed by the medical device 3 as being having a low diagnostic value 82 and as being negative for the specific disease 4. Other upper end values of the low diagnostic values 82 could be 75%, 85%, 90% and 95% of the value for the high diagnostic value 78.

Some embodiments of the present medical device 3 summarize or compile the syndrome elements 42 of each classic syndrome 18 into a database 40 or library or libraries. A plurality of libraries may be used e.g. to separate classic stroke syndromes 18 from classic stroke mimic syndromes 24. In such embodiments of the present medical device 3, the front-end patient interface 64 of the device could collect the initial symptom 6 reported by the patient 22, which the medical device 3 then could use to identify classic syndromes 18 from the libraries containing that initial symptom 6. Then, the medical device 3 could assess the distribution of other syndrome elements 42 from the selected classic syndromes 18, ranking the syndrome elements 42 based on the number of times they are found in the selected classic syndromes 18. As a next step, the medical device 3 could then query the patient 22 about the presence or absence of a most frequently encountered syndrome element 42. Based on the response from the patient 22, the subset of classic syndromes 18 selected for including the initial symptom 6 is reduced, leaving only their classic syndromes 18 that also contain the most common syndrome element 42. This process repeats until a single classic syndrome 18 is left remaining, or until a small group of classic syndromes 18 remains possible wherein all the remaining classic syndromes 18 are of a single type, e.g., classic stroke syndromes 18, at which time a diagnosis 20 can be delivered to the patient 22 and/or healthcare providers via a medical device output or via the network. In other embodiments, the patient 22 can be queried with an uncommon syndrome element 42 present in only a few or one of the initially selected classic syndromes 18 as a means of reducing the number of possible classic syndromes 18. In still other embodiments, examination findings 10 or other data inputs 14 may be used to select amongst, and exclude, various classic syndromes.

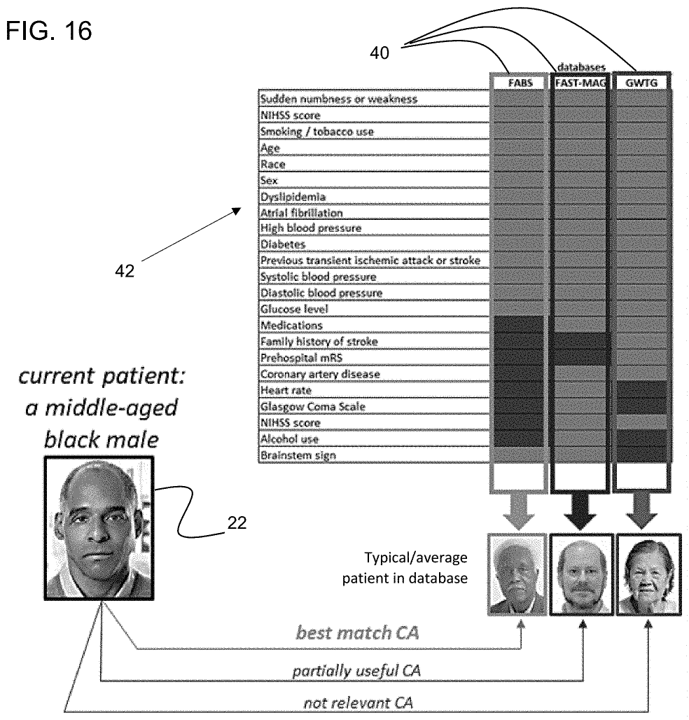

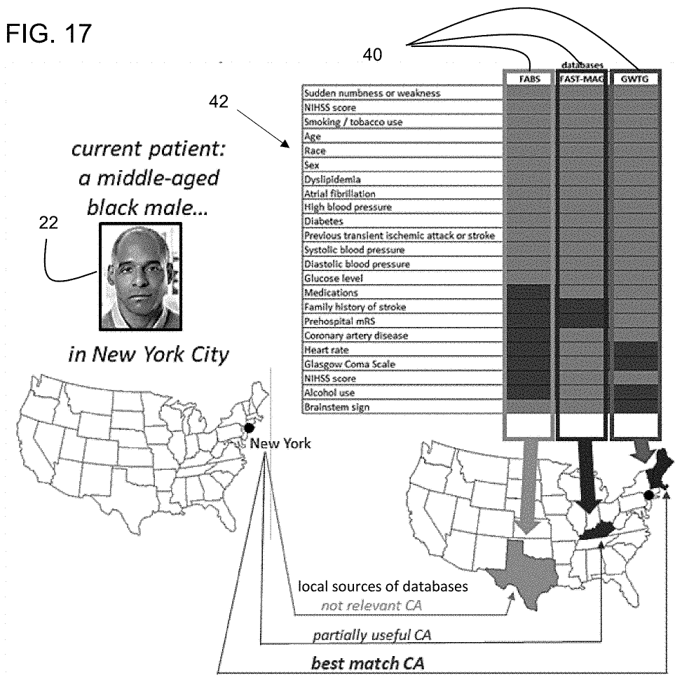

Referring to FIGS. 16 and 17, further embodiments are shown for selective or targeted use of databases 40 and computational algorithms 2 trained on certain databases 40. In this figure, the lighter grey cells indicate values/data inputs 14 are present in that database 40, and the darker grey cells indicate no values are present for the different elements of the different databases 40. For example, in the embodiment shown, the FABS database 40 is used and values are present for glucose level, but not for medications. Databases 40 may be chosen to use that are particularly relevant to the patient 22 under evaluation 30 by the device based on demographic features (age, sex, race, medical history, geography, etc.). Two examples are given in the figures. In FIG. 16, non-geographic/personal demographic feature similarities are used to choose a database 40 and its associated computational algorithm(s) most relevant to a patient. In FIG. 17, geographic similarities (e.g., geographic proximity) are used to choose the database 40 and its associated computational algorithm(s) most relevant to a patient. The demographic similarity or relevance of the database 40 to the patient 22 under evaluation 30 may cause the medical device 3 to employ computational processes tuned to or adapted for a certain database 40, even though that particular database 40 has less data values than another database 40.

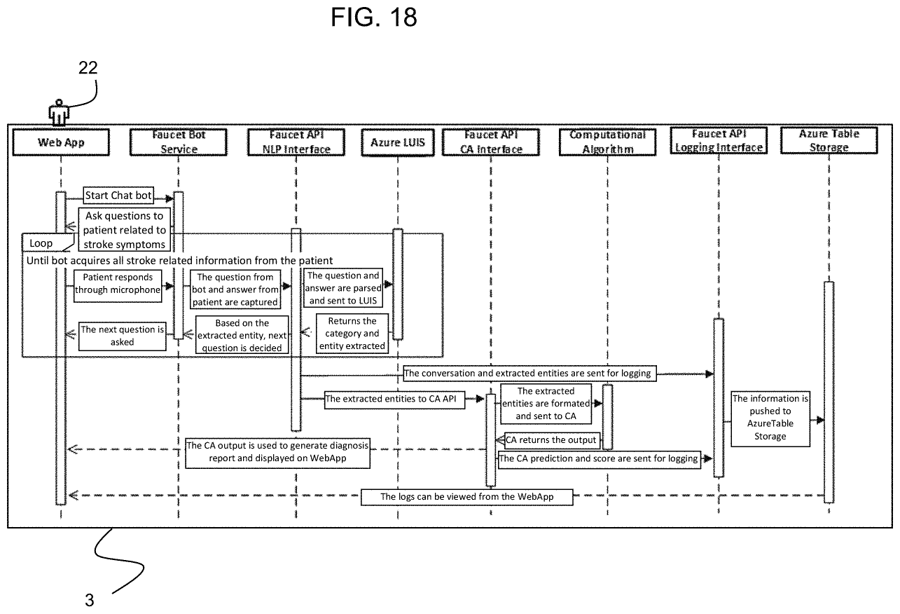

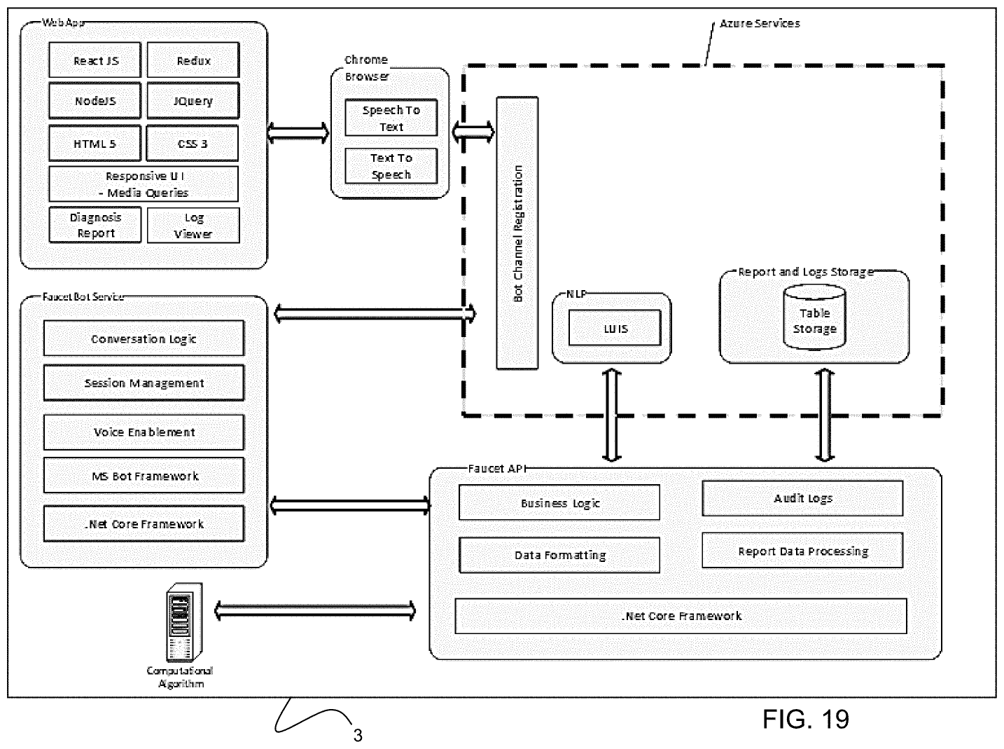

Referring to FIGS. 18 and 19, further embodiments are shown for utilization of the medical device 3 to diagnose diseases 4. In these embodiments, Microsoft® services (e.g., Azure®, LUIS®) are used as examples of e.g., functions or capabilities, but are considered to be exemplary of other similar services that may be used, for example from other companies such as Amazon®, Google®, IBM®, and others.

In further embodiments, the medical device 3 may be integrated into one or more domestic apparatuses 84, or home fixtures or appliances, such as a mirror, clock, refrigerator, sink, toilet, chair, bed, television, microwave oven, or electric light, including a light fixture attached to the ceiling, for example. Preferably the domestic apparatus 84 is one that a patient 22 will interact with or be in proximity to on a regular or frequent basis. The domestic apparatus 84 would preferably be connected or connectable to a network, such as WIFI, Bluetooth, cellular, and/or internet of things, and include the sensors 62 to passively or interactively record the condition of the patient 22, such as one or more microphones, cameras, and thermal cameras or thermometers, electrocardiography sensors, photoplethysmography sensors to measure heart rate and/or electromagnetic pulse monitor, for example, and potentially other input devices 76 to interact with the patient 22. The domestic apparatus integrated medical device 3 could have output elements 38, such as screens, lights, and speakers, for example, to prompt the patient 22 with lights, sounds, or spoken questions to elicit responses or otherwise interact with the patient 22. In embodiments where the medical device 3 monitors for patient reported perceptions 6 or physical signs 10 of stroke, for example, the camera could capture video to determine if there is drooping of one part of the face or body compared to the other. Speakers could capture and determine if there is any slurring or an increase in slurring when the patient 22 speaks, or the disruption of language grammar, syntax, or content. This could also be in reply to the medical device issuing a “good morning” greeting to the patient 22 either in spoken words via a speaker or written text via an interface 64, for example. Additionally, the medical device 3 could periodically or consistently monitor the patient 22's speech and if slurred speech is detected, could automatically trigger an evaluation 30 and/or send an alert 32 to care givers, paramedics, or other emergency personnel, and/or a central server or other monitor. The camera could also detect if there is a limp in the patient 22's gate. If the patient 22 has high risk factors for stroke, the medical device 3 could screen regularly for initial warning symptoms 6 or sign, and automatically trigger an evaluation 30 and/or send an alert 32 if an initial warning symptom 6 or sign 10 is detected. The medical device 3 may also instruct a stop-gap treatment for the patient 22 or both instruct and deliver a stop-gap treatment for the patient 22 if an initial warning symptom 6 is detected, or if a diagnosis 20 of a disease 4 is made. In the example of cardiac ischemia or transient ischemic attack, the device could instruct the patient 22 to take an aspirin while waiting for medical professionals to arrive. In further embodiments, the medical device 3 could dispense an aspirin or other appropriate emergency medication for the specific disease 4 diagnosed. The medication could be dispensed from a reservoir containing medication for one or more different diseases. The medications could be in containers or bags, color coded, numbered, named, or otherwise labeled, so that if multiple types of medications were made accessible to the patient 22, the appropriate medication would be clearly indicated. For example, the medical device 3 could state “You have been diagnosed has likely to be suffering from a transient ischemic attack, please take one aspirin—from the blue bag with the letter “A” in the pharmaceutical bin.” The medical device 3 may be self-activating. The medical device 3 is anticipated to be used in nursing and long-term care facilities, and for elderly home residents, for example.

The medical device 3 will preferably collect information with direct or indirect interaction with patient 22, but also with other sources, like third parties 74, such as witnesses of accident, nursing home workers, family members, medical personnel, and patient 22 hospital/medical records. If there are inconsistencies in the data entered, the medical device 3 could flag the inconsistencies and alert 32 a physician 28 and/or the medical device 3 could query the sources of the discordant information and others for clarification. For example, if a patient 22 inputted that a symptom 6 started six hours ago and a nurse inputted that the symptom 6 started two days ago, the inconsistency could be flagged by the medical device 3, and then the physician 28 or the medical device 3, for example, could determine what is true by further questioning of the sources of the discordant information. Additionally, or alternatively, the answers the various individuals give could be weighted 16 for accuracy based on the veracity or accurateness of other answers given by that individual 22 for that given question or for all questions posed to the individual 22 or for the veracity and accurateness of answers given by groups that the individual belongs to, for example nurses or ambulance personnel, or individuals working at a specific hospital.

In some embodiments of the medical device 3, the medical device 3 will only speak with and visualize the patient 22 as means of obtaining diagnostic information on the patient 22. In other, more preferred embodiments, the medical device 3 will be able to interact with a plurality of third parties 74 (individuals besides the patient 22), and other sources of information relevant to the patient 22, for example, multiple witnesses to an accident in which the patient 22 was injured, the patient's 22 family members or healthcare providers, and sources of the patient's 22 medical records. In some embodiments, the medical device 3 is comprised of a plurality of device units, one of which may be based within an ambulance vehicle 72 and others which may be small, handheld devices capable of transmitting information, such as voice, image, and inputted data information from a user (including a third party 74) to the data collection processes of the medical device 3. The handheld units can then be given by ambulance crewmembers to people 74 at the emergency site, allowing them to interact with the medical device system 3, with the separate units querying the people 74 about needed information relevant to the patient 22's condition, while the ambulance crewmembers see to the patient 22 or transport the patient 22 to the hospital. A benefit of having a separate medical device unit is that it could be already connected to a network with the rest of the medical device system 3, and already be loaded with questions to ask, at the time it is provided to the third party user 74—a key benefit when time is of the essence. In other embodiments, the separate medical device unit connects directly into communication-capable devices already available to the third parties 74, such as their personal cell phones or other computing devices 70, through which information pertinent to the patient's 22 diagnosis 20 can be queried and conveyed. In preferred embodiments of the invention, the medical device 3 will collect information on the patient 22 from multiple sources including the patient, third parties, and/or medical records in a parallel, simultaneous, or overlapping manner.

Paramedics could leave the one or more, preferably small handheld unit embodiments of the medical device 3 with third parties 74, such as witnesses or family members, especially if the patient 22 is unconscious upon the paramedics' arrival. The medical device units would present questions, input answers, and send data to the medical device system mainframe via wireless network for analysis, storage, and diagnostic decision making. This could be relayed to a physician 28 at a destination hospital and/or the paramedics conveying the patient 22 to the destination hospital. This could save time and increase quality of information. In one embodiment, the small handheld medical device unit could be put in the mail after the third parties 74 finish entering data into them for return and reuse.

In a further embodiment, a program for asking the third parties' information could be downloaded onto smart phones or computing devices 70 of the third parties 74, or the third parties 74 could be taken to a website that asks information from the third parties about the patient 22. The information would be sent via a network to a medical device central server to be compiled and analyzed and then sent to a medical professional 28 or diagnostic machine. Alternatively, the information could be sent to a medical professional 28 directly, or indirectly to be compiled and analyzed.

In a further embodiment, each or one or more patient 22 that is diagnosed as either having, not having, or being inclusive of having a particular disease 4, based on different data inputs 14, could be followed up for outcomes data. If the patients 22 received a definitive diagnosis 20 in the hospital or other medical care center by a physician 28 or medical professional, that diagnosis 20 could be used to create a post-use patient 22 database 40 to increase the accuracy of the computational algorithms 2 used by the medical device. That is, using for example, backpropagation and other analysis and computation, the accuracy of diagnoses 20 of past users 22 of the medical device 3 may be used in training and/or updating the computational algorithms 2 and determining the weighing 16 and choice of data inputs 14, including varying based on demographic features, and biases of the activation/transfer functions of nodes 54 as needed, in determining the diagnosis 20 of current users 22 of the medical device 3. In preferred embodiments, a portable computing and/or communication device is provided to the patient 22 during and after hospitalization which is capable of providing information about the patient's chronic condition to a database 40. In still other embodiments, the portable computing and/or communication device provided to the patient 22 can be used to monitor the patient 22's condition and/or summon emergency medicine services.

The presently claimed invention illustratively disclosed herein suitably may explicitly be practiced in the absence of any element which is not specifically disclosed herein. While various embodiments of the presently claimed invention have been described in detail, it is apparent that various modifications and alterations of those embodiments will occur to and be readily apparent those skilled in the art. However, it is to be expressly understood that such modifications and alterations are within the scope and spirit of the presently claimed invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherein, I claim:

1. A medical device comprising:
a memory;
a processor communicably coupled to the memory, and
the processor configured to execute instructions to:
evaluate one or more patient data inputs relating to a first specific disease;
compare the one or more data inputs to a set of values from at least one database using at least one computational algorithm;
train the at least one computational algorithm for estimating the diagnosis of the patient based on the first specific disease;
determine a first diagnostic score for the patient for the specific disease using the at least one computational algorithm;
diagnose the patient as having the specific disease when the first diagnostic score for the first specific disease is above a first value; and
provide the diagnosis for the patient as an output.

2. The medical device of claim 1 wherein the processor is further configured to execute instructions to diagnose the patient as not having the specific disease when the diagnostic score is below a second value, with the second value being below the first value.

3. The medical device of any claim 1 wherein the processor is further configured to execute instructions to diagnose the patient as inconclusive when the diagnostic score is between the first value and the second value.

4. The medical device of claim 1 further comprising one of a mirror measuring at least one square foot of reflective area, a clock, a refrigerator, a sink, a toilet, a chair, a bed, a television, a microwave oven, a floor standing or counter electric light, and a light fixture attached to the ceiling.

5. The medical device of claim 1 further comprising straps to allow the medical device to be worn on the wrist.

6. The medical device of claim 1, wherein the data inputs are one of demographic data, symptoms, medical history elements, examination findings, and diagnostic testing results, or some combination thereof.

7. The medical device of claim 1, wherein the data inputs are one of automatically acquired by the medical device and are received from one of the patient, a third party, and both the patient and the third party.

8. The medical device of any claim 1 wherein the medical device initiates interaction with the patient with a spoken prompt.

9. The medical device of claim 1, wherein the likelihood of diagnosis of a specific disease is increased when a positive symptom of that disease is present, and the likelihood of the diagnosis of the specific disease is decreased when separate symptom of a mimic disease is present.

10. The medical device of claim 1, wherein the first specific disease is one of a neurological abnormality,
heart failure,
asthma,
a myocardial infarction, and
an infection.

11. The medical device of claim 1, where the specific disease is a neurological abnormality and the neurological abnormality is one of acute stroke, transient ischemic attack, seizure, demyelinating diseases, multiple sclerosis, traumatic brain injury, and brain tumor, or some combination of each.

12. The medical device of claim 1, wherein the medical device automatically evaluates the patient for an initial sign of one or more of the specific diseases of conditions, and automatically triggers a more comprehensive evaluation when the initial sign is detected.

13. The medical device of claim 12, wherein one of
abnormal body temperature is assessed as an initial sign of infection;
one of alterations in gait, speech, and extremity movements, or some combination thereof are assessed as initial signs of a neurological abnormality;
one of breathing rates, interruptions in speech, and both are assessed as initial signs of worsening heart failure;
one of shortness-of-breath, labored breathing, and both are assessed as initial signs of an impending or actual asthma attack; and
one of gripping the chest, facial expressions indicative of pain, rapid breathing, flushing, and/or sweatiness, or some combination thereof are assessed as initial signs of myocardial infarction.

14. The medical device of claim 1, when the device diagnoses the patient as having the first specific disease of condition, the medical device one of
determines an appropriate medication to be taken by the patient and informs the patient, and
determines an appropriate medication to be taken by the patient, informs the patient, and then dispenses the medication directly also.

15. The medical device of claim 1 wherein the processor is further configured to execute instructions to evaluate the patient for a likelihood of a second disease that is a mimic to the first disease, and a diagnosis of the first disease is accompanied by an alert when the likelihood of an alternative diagnosis of the mimic condition is above one of 25%, 50%, 75%, and 90%.

16. The medical device of claim 1, wherein the at least one computational algorithm includes one or more of an artificial neural network, a support-vector machine (SVM), a Nu-SVM, a linear SVM, a Naive Bayes (NB) algorithm, a Gaussian NB, a multinomial NB computation algorithm, a multiclass SVM, a directed acyclic graph SVM (DAGSVM), a structured SVM, a least-squares support-vector machine (LS-SVM), a Bayesian SVM, a transductive support-vector machine, a support-vector clustering (SVC), a classification SVM Type 1 (C-SVM classification), a classification SVM Type 2 (nu-SVM classification), a regression SVM Type 1 (epsilon-SVM regression), and a regression SVM Type 2 (nu-SVM regression).

17. The medical device of claim 1, wherein the processor is further configured to send the diagnosis to a medical facility via a wired or wireless network, and the medical device further comprises means to communicate the diagnosis via the network.

18. The medical device of claim 1, wherein the processor is further configured to execute a second computational algorithm to determine a second diagnostic score for the patient when the first diagnostic score is determined to be one of
below the first value,
below the first value and above the second value, and
below both the first value and the second value.

19. The medical device of any claims 1-18, wherein the processor is further configured to
execute a plurality of computational algorithms, each using data from a plurality of databases,
determine a diagnostic score for the patient for the first specific disease for each of the computational algorithms, and
diagnose the patient as having the specific disease when one of a majority and all of the computational algorithms' diagnostic scores for the specific disease is above the first value.

20. A medical device comprising:
a memory;
a processor communicably coupled to the memory, and
the processor configured to execute instructions to:
evaluate one or more patient data inputs relating to a first specific disease;
compare the one or more data inputs to a set of values from at least one database using at least one computational algorithm;
train the at least one computational algorithm for estimating the diagnosis of the patient based on the first specific disease;
determine a first diagnostic score for the patient for the specific disease using the at least one computational algorithm;
diagnose the patient as having the specific disease when the first diagnostic score for the first specific disease is above a first value; and
provide the diagnosis for the patient as an output;
wherein the processor is further configured to
input one or more syndrome elements encompassed by a historical definition of a classic syndrome related to the first specific disease;
allocate syndrome elements points proportionate to a known or documented prevalence in a population of patients confirmed to have the classic syndrome;
determine if the patient has the syndrome elements;
calculate a probability of diagnosis of the patient having the classic syndrome as a ratio of total number of points representing the syndrome elements identified for the patient divided by a total number of points of all the syndrome elements encompassed by the historical definition of the classic syndrome, and
input the probability of diagnosis of the classic syndrome as a data input relating to the first disease;
wherein the first computational algorithm and second computational algorithm are part of a plurality of computational algorithms that refine a diagnosis for the patient in a serial manner;
wherein the first computational algorithm bases its calculations upon a common set of data inputs from multiple databases, and wherein the second computational algorithm bases its calculations upon all the data inputs from a single database; and
wherein the second computational algorithm is chosen from a plurality of computational algorithms each basing its calculations upon all the data inputs from separate databases, and
wherein the second computational algorithm selection is based on similarity between the patient's data inputs and the data inputs of the database used by the second computational algorithm.
